(19) United States
(12) Patent Application Publication
(10) Pub. No.: US 2022/0195382 A1
(43) Pub. Date: Jun. 23, 2022

(54) A NOVEL HUMAN-MATERIAL-BASED PLATFOM TECHNOLOGY FOR TISSUE ENGINEERING

(71) Applicant: THT Biomaterials GmbH, Vienna (AT)

(72) Inventors: Johannes HACKETHAL, Vienna (AT); Heinz REDL, Vienna (AT); Andreas Herbert TEUSCHL, Vienna (AT)

(21) Appl. No.: 17/600,591

(22) PCT Filed: Apr. 3, 2020
(86) PCT No.: PCT/EP2020/059514

§ 371 Date: Sep. 30, 2021

Related U.S. Application Data
(63) Continuation-in-part of application No. PCT/EP2019/058534, filed on Apr. 4, 2019.

(30) Foreign Application Priority Data
Apr. 4, 2019 (EP) PCT/EP2019/058534

(51) Int. Cl. C12N 5/00; C12N 5/077; C12N 5/071

(52) U.S. Cl. CPC C12N 5/0068; A61K 35/00; C12N 5/069; C12N 5/0656

(57) ABSTRACT

The present invention relates to a biologically active placenta-derived liquid human substrate (hpS) comprising extracellular matrix (ECM) proteins, cytokines and growth factors and use thereof. The present invention also provides methods of producing a composition comprising biologically active placenta-derived liquid human substrate (hpS).

### FIELD OF THE INVENTION

The present invention relates to the field of regenerative medicine. More particularly, the invention pertains to compositions comprising biologically active human substrate and to methods for producing such compositions.

### BACKGROUND ART

Over 500 million people worldwide would currently benefit from pro- or anti-angiogenesis treatments. Numerous pathological entities or surgical inventions could benefit from therapeutic stimulation of new blood vessel formation. Wound healing, myocardial ischemia, plastic surgery or cancer research is just a few of many situations that could be improved through a new or regenerated blood vessel system. Hence, the success of many current therapies in regenerative medicine requires the ability to create stable, hierarchically organized vascular networks within the engineered or regenerated tissues. In any tissue or scaffold of relevant size, viable cells need to be within a distance of maximal 200 μm of pre-existing blood vessels (the diffusion limit of oxygen and nutrients within tissues), to stay alive. Therefore, therapeutic stimulation of new blood vessel formation (neovascularization) is a key objective of research in tissue engineering and regenerative medicine (TERM).

Currently, there is a broad variety of choices when selecting scaffold biomaterials for TERM. Various synthetic or natural polymers were already tested as scaffold materials for 3D in vitro vasculogenesis and angiogenesis research. However, the success rate of complete vessel maturation and therefore the clinical relevance of most of these biomaterials is limited and associated with various bottlenecks. Generally, most models contain few polymers, e.g., poly-ethylene-glycol (PEG) or collagen-1), one distinct cell type (e.g. HUVEC), and one bioactive component, e.g., vascular endothelial growth factor (VEGF). Therefore, these one-component models often reflect distinct effects in the cascade of neovascularization, but as they do not adequately mimic the natural diversity of native tissue, they do not successfully induce vessel maturation, which is however essential for vascularized biomaterials at larger scales. For instance, synthetic polymers are generally cheap, well defined and highly processable, however they are inert and the vast majority of synthetic polymers do not exhibit cell-interactive properties. In contrast, the heterogenic mix of ECM proteins, processed from tissues, are the most natural scaffolds. In nature, neovascularization is orchestrated by different molecular mechanisms of different kinds of proteins within the ECM, that is in total composed of over 300 different proteins, proteoglycans and signaling molecules in humans. The ECM is nature's own multifunctional scaffold, thus, the ideal environment for human cells is provided by the human natural ECM. ECM has a profound impact on the behavior of all eukaryotic cells, acts as the reservoir for growth factors and exerts fundamental control over angiogenesis in all neovascularization stages. ECM modulates a wide range of fundamental mechanisms in development, function and homeostasis of all eukaryotic cells. Therefore, biomaterials extracted from naturally occurring ECM have received significant attention in TERM.

A prominent example is Matrigel, a heterogeneous substrate extracted from tissues derived from Engelbreth-Holm-Swarm (EHS) tumor in mouse models, which represent the gold standard for many in vitro vasculogenesis and in vivo angiogenesis studies in research.

Matrigel is also a frequently-used substrate for hepatocyte-toxicology studies, cancer research, or stem cell studies. In November 2018, the search term “Matrigel” listed over 10,000 publications on the PubMed database, which proves the evolving interest in this material over the last decades. Major components of Matrigel are laminin-111 (around 60%) and collagen-4 (around 30%), which form basement-membrane-like structures at 37° C. Matrigel is described to additionally contain entactin (nidogen), heparan sulfate proteoglycan, and six growth factors (basic fibroblast growth factor (bFGF; <0.1-0.2 pg/mL), epidermal growth factor (EGF; 0.5-1.3 ng/mL), insulin-like growth factor-1 (IGF-1; 11-24 ng/mL), platelet-derived growth factor (PDGF; 5-48 pg/mL), nerve growth factor (NGF; <0.2 pg/mL) and transforming growth factor-β1 (TGF-β1; 1.7-4.7 ng/mL).

However, the major drawback of Matrigel is that it is not intended for clinics, due to its xenogenic tumorigenic origin. Additionally, production of Matrigel requires the sacrifice of large numbers of animals.

Furthermore, many xenogenic biomaterials are still associated with immunological responses in up to 5% of all patients harbor the risk of xenogenic pathogen contamination and potential disease transmission. Thus, their use in large clinical studies is controversially debated. In addition, many xenogenic proteins are known to have a lower clinical performance when compared to human proteins. Hence, ECM extracted from human origin is regarded as the best option for the creation of new medical products, because the ECM structures of donors and recipients are identical. Moreover, ECM biomaterials intended for human in vivo applications (e.g. filler) mostly aim to be decellularized, in order to lower immune responses provoked by foreign DNA remnants. A fully decellularized tissue is currently defined as ECM proteins with less than 50 ng/mL DNA dry tissue weight, DNA fragment size below 200 bp and the absence of visible cellular particles stained with hematoxylin and eosin, and DAPI″.[1]

WO2014165602 discloses methods and compositions, including a placental extract, for inducing and/or modulating angiogenesis. The placental extract is made by obtaining a sample from a human placenta, removing blood from the placental sample to produce a crude placental extract, mixing the crude placental extract with urea to solubilize the proteins present in the extract, removing remaining solids from the crude extract; dialyzing the urea-placental extract mixture to remove a substantial amount of the urea from the mixture to produce the human placental extract. However, the pro-angiogenic factor content is substantially low due to the use of high urea concentrations.

WO2017/112934 A1 describes a decellularized placental membrane and a placenta-derived graft comprising the decellularized placental membrane. US2016030635 discloses methods of producing extracellular matrix (ECM). The double dried ECM is provided as sheets which comprise between 70% and 95% collagen-1 and less than 1% laminin-111. A placenta-derived composition comprising placental tissue and one or more protease inhibitors is described in WO2017160804 A1. This placenta-derived composition is an acellular composition wherein the amount of various proteins is increased by the addition of protease inhibitors and whereas decellularized tissue is defined as ECM proteins with less than 50 ng/mL DNA dry tissue weight, DNA fragment size below 200 bp and the absence of visible cellular particles stained with hematoxylin and eosin.

Therefore, there is still the need for improved biologically active human substrate compositions which facilitate the creation of new vascularized tissues and thus are able to replace injured tissues and/or organs.

### SUMMARY OF INVENTION

It is the object of the present invention to provide an improved liquid composition comprising biologically active human substrate with an increased content on basal membrane proteins (laminin-111, collagen-4) and pro-angiogenic factors and a decreased content on stroma proteins (collagen-1). The object is solved by the subject matter of the present invention.

According to the invention, there is provided a liquid composition comprising biologically active liquid human substrate from placenta (hpS) with an increased content of pro-angiogenic growth factors when compared to basement membrane matrix for cell growth and differentiation. Specifically, liquid the placenta substrate is obtained by a treatment with a non-denaturizing protein solubilization agent and exhibits an increased content of pro-angiogenic growth factors when compared to basement membrane matrix for cell growth and differentiation.

Further is provided a liquid placenta-derived substrate (hpS) which comprises basal membrane proteins with increased content of cytokines and growth factors when compared to Matrigel, and obtainable by a treatment with a non-denaturizing protein solubilization agent. In one embodiment of the invention the liquid placenta-derived substrate as described herein, is obtained by a method wherein the placenta material is treated with NaCl solution, preferably with a Tris 0.5 M NaCl buffer.

In one embodiment the biologically active placenta-derived liquid substrate (hpS) comprises extracellular matrix (ECM) proteins with increased content of cytokines and growth factors. Specifically, the content of cytokines and growth factors is increased when compared to Matrigel.

In certain embodiments, hpS comprises laminin-111 and one or more of collagen-4, fibronectin and glycosaminoglycans.

One embodiment of the invention relates to the liquid composition as described herein, wherein the pro-angiogenic growth factors comprise of angiogenin (ANG), angiostatin (PLG), basic fibroblast growth factor (bFGF), tissue inhibitor of metalloproteinases (TIMP), growth regulated protein (GRO), matrix metalloproteinase (MMP), angiopoietin (ANGPT), platelet endothelial cell adhesion molecule (PECAM), Leptin, interleukins (IL), RANTES (CCL5), tyrosine kinase-2 (TIE-2), urokinase plasminogen activator (uPAR), tumor necrosis factor-alpha (TNF-α), epidermal growth factor (EGF), granulocyte colony stimulating factor (G-CSF), monocyte chemotactic protein (MCP), interferon inducible T-cell α chemokine (I-TAC), monocyte chemotactic protein (MCP), epithelial neutrophil activating peptide 78 (ENA-78), 1-309 (CCL1), endostatin, platelet-derived growth factor (PDGF), vascular endothelial growth factor (VEGF), interferon gamma (IFN-γ), insulin-like growth factor 1 (IGF-1), placental growth factor (PLGF), granulocyte macrophage colony stimulating factor (GM-CSF), transforming growth factor (TGF), thrombopoietin (THPO).

In one embodiment of the invention, the liquid composition as described herein comprises increased levels of ANG, PLG, GRO, MMP-1/9, PECAM-1, IL-1 alpha, IL-1 beta 2/4/6/8/10, TIE-2, TNF-alpha, MCP-1/3/4, IFN-gamma, PLGF, TGF-beta1, VEGF, when compared to urea extracts.

One embodiment of the invention relates to the liquid substrate as described herein, wherein the extracellular matrix (ECM) proteins are selected from the group consisting of basal membrane proteins and proteins from blood lineage. The basal membrane proteins may be fore example collagen-4 and laminin 111 and the protein from blood lineage may be for example thrombin. The content of laminin 111 may be for example up to 90%, or up to 85%, or up to 80% of the total protein content. The content of collagen-4 may be about 10% of the total protein content. In one example the collagen-1 content in the liquid substrate is less than 0.1% of the total protein content.

A further embodiment of the invention relates to a liquid composition as described herein, wherein the protein content is in the range of 1.0 to 2.0 mg/mL, or 1.5 to 1.9 mg/mL, or 1.7 to 1.8 mg/mL. In a specific embodiment the protein concentration of the composition is of about 1.75 mg/mL.

One embodiment of the invention relates to a composition as described herein, which further comprises one or more compounds selected from the group consisting of antimicrobial agents, analgesic agents, local anesthetic agents, anti-inflammatory agents, immunosuppressant agents, anti-allergenic agents, enzyme cofactors, essential nutrients, growth factors, human thrombin cytokines, and chemokines, or combinations thereof. A further embodiment relates to the liquid substrate as described herein, comprising additionally one or more antimicrobial agents.

A further embodiment of the invention relates to the liquid composition as described herein, wherein the substrate does not gel at temperatures up to 37° C.

In one embodiment of the invention the liquid composition as described herein is solidified by the addition of fibrinogen.

One embodiment of the invention relates to the liquid composition as described herein, further comprising natural polymers or synthetic polymers.

One embodiment of the invention relates to a process for preparing a liquid composition comprising a biologically active human substrate comprising the steps of:
a. providing a sample from human placenta;
b. removing blood from said sample to obtain a crude extract;
c. solubilizing proteins in said crude extract using a Tris NaCl buffer separating solid materials from the solubilized protein extract mixture;
d. dialyzing the solubilized protein extract; and
e. obtaining the biologically active human placenta substrate.

A further embodiment of the invention relates to the method as described herein, wherein the extraction step is carried out using at least 0.2, 0.3, 0.4, 0.5, 0.6, 0.7, 0.8, 0.9, 1, 2, 3, 4, 5, or 6 M Tris-NaCl buffer. In one example, the method is carried out with Tris 0.5 M NaCl buffer

A further embodiment of the invention relates to the method as described herein, wherein the extraction step is carried out in the absence of urea, guanidine-HCl, sodium dodecyl sulfate (SDS), Triton X-100 or enzymatic digestives, such as pepsin, and protease inhibitors or animal products.

A further embodiment of the invention relates to the method as described herein, wherein the biologically active human substrate is admixed with a natural and/or synthetic polymer.

A further embodiment of the invention relates to the use of the placenta substrate (hpS) as coating material or scaffold material in biological assay.

One embodiment of the invention relates to the use of the substrate in a variety of clinical applications.

A further embodiment of the invention relates to the use of the placenta substrate (hpS) for 2D and 3D in vitro neovascularization studies. One embodiment of the invention relates to the use wherein in said studies human malignant and normal cells derived from exoderm, mesoderm or endoderm lineage are employed.

A further embodiment of the invention relates to the use of the placenta substrate (hpS) as a cell culture medium supplementation, whereas said hpS is added to a cell culture medium.

A further embodiment of the invention relates to the use of the placenta substrate (hpS) as described herein, wherein the cell culture medium is a defined minimal essential cell culture medium.

A further embodiment of the invention relates to the use of the placenta substrate (hpS) for 2D or 3D in vitro toxicology-, stem cell-, spheroid- or organoid studies.

### BRIEF DESCRIPTION OF DRAWINGS

FIG. 1: Flow chart for the isolation of human placenta substrate (hpS) from term placenta (1). After basal tissue collection (2), main blood components were removed by subsequent homogenization and centrifugation steps (3). Finally, hpS was isolated by salt precipitation using a Tris 0.5 M NaCl buffer (4), centrifugation (5) and PBS dialysis (6) to yield hpS.

FIG. 2 depicts that hpS contains a heterogenic mixture of proteins. (A) Protein quantification of hpS (n=6). (B) CyQuant DNA quantification showing DNA content of native unprocessed placenta tissue, hpS Tris-urea and hpS Tris-NaCl isolates, respectively (n=6). (C) DMB staining showing GAG content in native placenta tissue, hpS Tris-urea and hpS Tris-NaCl, respectively. (n=6) (D) Coomassie blue stained 3-8% SDS-polyacrylamide gel (1) showing Marker, Matrigel or hpS Tris-NaCl and a 12% SDS-polyacrylamide gel (2) showing hpS Tris-NaCl, a second precipitation and a Marker. Representative immunoblots showing (E) collagen-1, (F) collagen-4, and (G) laminin-111 content in Matrigel, hpS Tris-urea and hpS Tris-NaCl.

FIG. 3: Angiogenic profile of hpS Tris-urea and hpS Tris-NaCl in normalized intensity to the positive control, standardized IgG (n=3). Proteolytic enzymes [metalloproteinases (MMP-1/9)]. Immune related cytokines [interleukins (IL-1α/β,2,4,6,8,10), interferon-γ (IFN-γ)]. Growth factors [basic fibroblast growth factor (bFGF), vascular endothelial growth factor receptor (VEGFR2/3), tumor necrosis factor-α (TNF-α), epidermal growth factor (EGF), granulocyte-colony stimulating factor (G-CSF), platelet-derived growth factor (PDGF), vascular endothelial growth factor (VEGF-A/D), insulin-like growth factor 1 (IGF-1), placental growth factor (PLGF), granulocyte-macrophage colony-stimulating factor (GM-CSF), transforming growth factor-β1 (TGF-β1), thrombopoietin (THPO)]. Angiogenesis related proteins [angiogenin (ANG), angiostatin (PLG), tissue inhibitor of metalloproteinases (TIMP-1/2), growth-regulated oncogene (GRO), angiopoietin (ANGPT1/2), PECAM-1, leptin, rantes, urokinase plasminogen activator (uPAR), tyrosine kinase-2 (TIE-2), monocyte chemoattractant protein (MCP-1/3/4), I-TAC, epithelial neutrophil-activating peptide 78 (ENA-78), 1-309, endostatin].

FIG. 4: VEGF ELISA showing VEGF content of Tris-urea and Tris-NaCl extracted substrates, respectively (n=3).

FIG. 5: Antimicrobial effects of hpS Tris-NaCl in two gram-negative strains (E. coli TOP10, E. coli MG1655) and two gram-positive strains (S. carnosus, S. capitits).

FIG. 6: 3D solidification of hpS. Various polymers were mixed with hpS to form stable 3D gels. As an example, hpS and fibrinogen was mixed without thrombin or aprotinin supplementation to gel at 37° C.

FIG. 7: HUVEC seeding density on hpS coated well plates in 2D. (A-C) Different HUVEC cell numbers were seeded on hpS coated wells for 2 days and the cell networks were analyzed (total/mean tubule length, junctions). The highest network complexity was observed when using 20,000 cells (=60,000 cells/cm², n=9). (D,E) CD31/DAPI and Ve Cad/DAPI staining of formed HUVEC networks (scale bar=200 μm), (F-H) Comparison of 3 substrates (hpS Tris-NaCl, Tris-urea, or Matrigel) using HUVEC cells. Asterix indicate statistical differences between hpS Tris-NaCl/Matrigel. No significant difference between hpS Tris-NaCl/hpS Tris-urea were observed (n=10).

FIG. 8: Single placenta substrate compared in 2D. (A) Microscopical images of HUVEC cell networks cultivated on hpS Tris-NaCl or Matrigel coated well plates for five days showing close-mesh HUVEC cell networks cultivated on hpS and wide-mesh HUVEC cell networks cultivated on Matrigel (scale bar=400 μm). (B) Analyzed characteristics of the 2D HUVEC cell networks (total/mean tubule length, junctions) cultivated on three different batches of hpS Tris-NaCl, extracted from three different organs, or cultivated on Matrigel (n=10).

FIG. 9: HUVEC/NIH3T3 fibroblast culture in 2D. (A) Fibroblasts spontaneously form cord-like structure when seeded on Matrigel, but not on when seeded on hpS Tris-NaCl (Scale bars=400 μm). (B) HUVEC cultivated on coated wells (extracted with a Tris-0.15 M NaCl buffer) showed a different phenotype when compared to HUVEC cultivated on hpS Tris-NaCl coated wells (extracted with a Tris-0.5 M NaCl buffer) after two days. (C) HUVEC form interconnected cell networks in the presence of hpS as a cell culture medium supplement, but not without hpS (Scale bars=400 μm).

FIG. 10: hpS to substitute FCS. (A) HaCaT MTT viability tests: A significant difference between FCS or hpS supplemented culture conditions was assessed after 5 days of culture (n=12). (B) HepG2 MTT viability tests: No significant difference between FCS or hpS supplemented culture conditions was assessed in the first 5 days of culture (n=4). (C) Different cell types were cultivated in medium supplemented with FCS or hpS (microscopic images 5 days after seeding, 100× magnification).

FIG. 11: hpS as 2D coating material. (A) NIH3T3 fibroblast MTT viability tests: A significant difference between Matrigel (MG) and hpS coated culture conditions was assessed after 5 days culture when using 150 μg/mL (n=16). (B) Proliferation of primary rat hepatocytes on uncoated, collagen-1 or hpS coated wells. Easz4you assay four hours after seeding showing significantly increased cell viability on hpS in comparison to collagen-1 or uncoated wells (n=20). (C) PC 12 cells cultivated on collagen-1, Matrigel or hpS at concentrations of 100 μg/mL after 2 days of culture (n=6). Scale bars=200 μm.

FIG. 12: 3D in vitro bioactivity of hpS. (A) HUVEC seeded in fibrinogen (Tisseel, Baxter) mixed with hpS Tris-NaCl or thrombin (0.4 U) for a total of 11 days (scale bars=400 μm). (B) SEM images of the clots (scale bars=10 μm). (C) Primary malignant colon organoids cultivated in Matrigel or a hpS/fibrin gel (5 mg/mL fibrin). Microscopical images after 5 days of culture, scale bar=200 μm.

FIG. 13: Table 1: Amino acid analysis of hpS Tris-NaCl (residues per 1.000 residues) compared to ECM proteins from literature.

### DESCRIPTION OF EMBODIMENTS

The present invention provides a composition comprising biologically active human substrate from placenta; hpS, with an increased content of pro-angiogenic growth factors when compared to basement membrane matrix and the composition is devoid of collagen-1. The composition is specifically useful for cell growth and differentiation.

Therapeutic stimulation of new blood vessel formation (neovascularization) would harbor major benefits for TERM. The success of many current therapies in regenerative medicine requires the ability to create and control stable vascular networks within the engineered or regenerated tissues. Therefore, the generation of vascularized tissue is currently one of the key challenges in TERM.

In order to promote vascularization for tissue engineering sustained delivery of growth factors effecting vasculogenesis and angiogenesis is a prerequisite for successful modulation of angiogenesis.

The present approach uses fractionation and separation techniques to obtain a complex composition of active human biomolecules isolated from the human placenta (hpS).

As a primary active site of angiogenesis, the placenta is one of the richest sources of pro-angiogenic factors. A number of pro-angiogenic factors have been identified, non-exclusive examples of which include angiogenin (ANG), angiostatin (PLG), basic fibroblast growth factor (bFGF), tissue inhibitor of metalloproteinases (TIMP), growth regulated protein (GRO), matrix metalloproteinase (MMP), angiopoietin (ANGPT), platelet endothelial cell adhesion molecule (PECAM), Leptin, interleukins (IL), RANTES (CCL5), tyrosine kinase-2 (TIE-2), urokinase plasminogen activator (uPAR), tumor necrosis factor-alpha (TNF-α), epidermal growth factor (EGF), granulocyte colony stimulating factor (G-CSF), monocyte chemotactic protein (MCP), interferon inducible T-cell α chemokine (1-TAC), monocyte chemotactic protein (MCP), epithelial neutrophil activating peptide 78 (ENA-78), 1-309 (CCL1), endostatin, platelet-derived growth factor (PDGF), vascular endothelial growth factor (VEGF), interferon gamma (IFN-γ), insulin-like growth factor 1 (IGF-1), placental growth factor (PLGF), granulocyte macrophage colony stimulating factor (GM-CSF), transforming growth factor (TGF), thrombopoietin (THPO).

The present disclosure provides compositions, wherein the pro-angiogenic growth factors are selected from the group consisting of angiogenin (ANG), angiostatin (PLG), basic fibroblast growth factor (bFGF), tissue inhibitor of metalloproteinases (TIMP), growth regulated protein (GRO), matrix metalloproteinase (MMP), angiopoietin (ANGPT), platelet endothelial cell adhesion molecule (PECAM), Leptin, interleukins (IL), RANTES (CCL5), tyrosine kinase-2 (TIE-2), urokinase plasminogen activator (uPAR), tumor necrosis factor-alpha (TNF-α), epidermal growth factor (EGF), granulocyte colony stimulating factor (G-CSF), monocyte chemotactic protein (MCP), interferon inducible T-cell α chemokine (I-TAC), monocyte chemotactic protein (MCP), epithelial neutrophil activating peptide 78 (ENA-78), 1-309 (CCL1), endostatin, platelet-derived growth factor (PDGF), vascular endothelial growth factor (VEGF), interferon gamma (IFN-γ), insulin-like growth factor 1 (IGF-1), placental growth factor (PLGF), granulocyte macrophage colony stimulating factor (GM-CSF), transforming growth factor (TGF), thrombopoietin (THPO).

The present disclosure provides compositions with increased levels of ANG, PLG, GRO, MMP-1/9, PECAM-1, IL-1 alpha, IL-1 beta 2/4/6/8/10, TIE-2, TNF-alpha, MCP-1/3/4, IFN-γ, PLGF, TGF-β1, VEGF, when compared to urea extracts.

Various synthetic or natural polymers were already tested as scaffold materials for 3D in vitro vasculogenesis and angiogenesis research. However, the success rate of complete vessel maturation and therefore the clinical relevance of most of these biomaterials is limited and associated with various bottlenecks. Generally, most models contain few polymers, e.g., poly-ethylene-glycol (PEG) or collagen-1, one distinct cell type (e.g., HUVEC), and one bioactive component, e.g., vascular endothelial growth factor (VEGF). Therefore, these one-component models often reflect distinct effects in the cascade of neovascularization, but as they do not adequately mimic the natural diversity of native tissue, they do not successfully induce vessel maturation, which is however essential for vascularized biomaterials planned for transplantation. For instance, synthetic polymers are generally cheap, well defined and highly processable, however they are inert and the vast majority of synthetic polymers do not exhibit cell-interactive properties. In contrast, the heterogenic mix of ECM proteins, processed from tissues, is the most natural scaffolds. In nature, neovascularization is orchestrated by different molecular mechanisms of different kinds of proteins within the ECM that is in total composed of over 300 different proteins, proteoglycans and signaling molecules in humans. The ECM is nature's own multifunctional scaffold, thus, the ideal environment for human cells is provided by the human natural ECM. ECM has a profound impact on the behavior of all eukaryotic cells, acts as the reservoir for growth factors and exerts fundamental control over angiogenesis in all neovascularization stages. ECM modulates a wide range of fundamental mechanisms in development, function and homeostasis of all eukaryotic cells. Therefore, biomaterials extracted from naturally occurring ECM have received significant attention in TERM. As a consequence, human-tissue extracted ECM is regarded as the best option for the creation of new medicinal products, because the ECM structures of donors and recipients are almost identical among species. Human placenta, a medical waste product in consistent quantity and quality, is described as a tissue with a strong pro-angiogenic potential. Placenta ECM proteins are free of any ethical conflicts. Placenta is globally and consistently available after birth for processing on large scales. This unique temporally human tissue harbors high amounts of various pro-angiogenic proteins. Various placenta-ECM-derived biomaterials have already been used as a biomaterial for in vitro and in vivo vasculogenesis and angiogenesis studies, and already integrated in routine clinical use. Placenta tissue is also reported to have very good antibacterial, anti-inflammatory and anti-scarring properties. Some human placenta ECM-extracted substrates such as Plaxentrex® (M/s Albert David, India), Laenec® (Japan Bioproducts Industry, Japan) or Melsmon Cell Revitalization Extract® (Melsmon Pharmaceuticals, Japan), which are mainly extracted by use of heat and pressure, have been successfully used for decades as a topical or injectable agent in clinical approaches related to wound healing, burn injuries, post-surgical dressings and bedsores, but their potential for neovascularization in tissue engineering is at least to our knowledge unknown. Probably, because Placentrex® for instance contains only fragments of fibronectin and some smaller peptides, glycosaminoglycans, lipids and polynucleotides, but it is not highlighted to contain any active pro-angiogenic factors that might have survived the heat-extraction.

In addition, allogenic transplantation of the human amnion (hAM) for clinical applications has already been successfully performed for over 100 years. Nowadays, it is also used for ophthalmology, wound healing and regenerative medicine purposes. In all these clinical studies, applications of placenta ECM components have been proven to be safe to patients. The present disclosure provides a composition as described herein, wherein the biologically active human substrate comprises extracellular matrix (ECM) proteins which are selected from basal membrane proteins, preferably laminin-111 or collagen-4.

Matrigel is originally extracted using a Tris 2 M urea buffer.[2] Various authors also used 2 M urea to isolate bioactive ECM from xenogeneic tissues.[3,4] Uriel and colleagues for instance used Tris 2 M urea to isolate pro-angiogenic ECM gels for in vitro studies from dermis or fat tissue, with an additional dispase treatment performed to lower the DNA content to a final yield of 183.7±10.2 ng/mL[4] This step could be easily integrated in our presented isolation method to significantly lower the remaining DNA in hpS as well, however, may have also an influence on its final bioactivity. Moore and colleagues used urea buffers ranging from 4 to 15 M, to isolate a pro-angiogenic protein fraction from human placenta.[4] However, urea is an endogenous product of protein and amino acid catabolism primary present in liver tissue, and, the cancerogenic potential of urea has also still not been adequately assessed, due to relatively few studies that have tested the toxicokinetics of exogenous urea in clinical studies to date. Due to all these issues, Tris 0.5 M NaCl buffers were used in our experiments to isolate hpS, which are reported to preserve higher amounts of angiogenic cytokines compared to Tris-urea buffers if used for the preparation of tissue isolates.

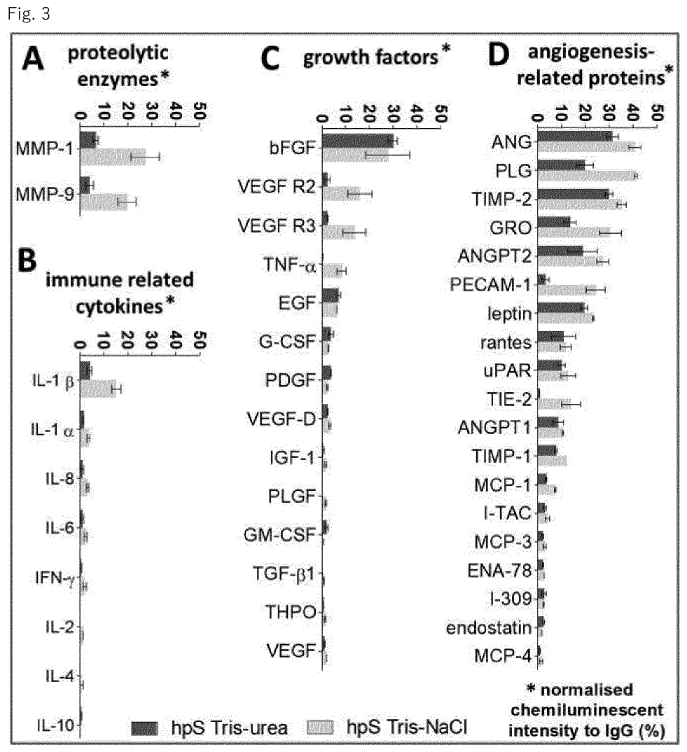

On average, 300-400 mL of liquid hpS were extracted from one single placenta weighing around 500 g. Hence, our substrate could be used as a coating, injected into tissues or soaked into any preexisting porous 3D materials for various cell culture applications. The total protein concentration of hpS using a Tris 2 M urea buffer was significantly higher when compared to the Tris 0.5 M NaCl buffer, which might be the result of the higher ionic density. For instance, Moore and colleagues used a Tris 4 M urea buffer to yield a similar protein content to Matrigel (around 15-20 mg/mL).[1,2] Hence, higher ionic densities yield higher amounts of extracellular matrix proteins. But in the same way, they also seem to lower the amounts of residual bioactive growth factors (see FIG. 3). No significant differences of GAGs were detected in both hpS extracts when compared to native tissues.

Although fewer extracellular matrix (ECM) proteins are isolated by the buffer solution with low salt concentration, surprisingly a higher balance of bioactive growth factors is obtained. Therefore, the protein content of the composition according to the present invention is in the range of 1.0 to 2.0 mg/mL, or 1.5 to 1.9 mg/mL, or 1.7 to 1.8 mg/mL, or the composition contains about 1.75 mg/mL protein.

Using SDS PAGE, a heterogenic variety of separate protein bands ranging up to around 500 kDa were found in hpS Tris-NaCl, which may represent an acceptable mimicry of the fully diversity of non-cellular physiologic human tissue (ECM), whereas Matrigel from tumors is composed of less proteins (mainly laminin-111). On Western blots, collagen-1 was only detectable in urea-enriched buffers (Matrigel, hpS Tris-urea), but not on hpS Tris-NaCl. On angiogenesis arrays, higher amounts of various angiogenesis related proteins was assessed using the isolation protocol based on a Tris 0.5 M NaCl buffer, when compared to the use of a Tris 2 M urea buffer, to extract hpS. Angiogenin, the most prevalent chemokine in hpS, was also the most prevalent chemokine using a Tris 4 M urea buffer in literature, but only relatively low levels of other angiogenic proteins were found.[2] Choi and colleagues used 0.5% SDS to extract ECM from human placenta and showed relatively high amounts of bFGF, TIMP-2, hepatocyte growth factor (HGF) or IGF binding proteins (IGFBP-1), but only relatively low levels of angiogenin were found.[5].

In this regard, beside angiogenin, a heterogeneous mixture of other angiogenic growth factors and chemokines led to the observed gfpHUVEC network formation on hpS. For instance, laminin-111 promotes angiogenesis in synergy with FGF-1 by gene regulation in endothelial cells. Leptin, an endocrine hormone, stimulates angiogenesis in synergistic effect with FGF. Another prominent example is VEGF, known to play fundamental roles in early phase of neovascularization (tip cell), whereas angiopoietin is associated to late stage neovascularization (maturation of blood vessels). hpS Tris-NaCl also contains thrombin, which upon mixing with fibrinogen can be used to form stable fully-human 3D fibrin scaffolds (clots). hpS Tris-NaCl has also antimicrobial properties dependent on the bacterial strain. The antibacterial effect was most prominent in S. carnosus, whose growth was almost completely inhibited by hpS Tris-NaCl. Interestingly, other strains were not affected by hpS Tris-NaCl. However, the underlying mechanism has not been investigated so far. The total amino acid analysis was used to identify the content of amino acids suitable for chemical crosslinking with other materials. The amino acid composition of hpS Tris-NaCl showed similar patterns like laminin-111, which was confirmed by Western Blot analysis, and displayed relatively high contents of amino acids with modifiable side groups (around 20 mol % NH₂/COOH residues) and therefore various chemical methods such as an anhydride strategies (e.g., norbornene anhydride), NHS activation (e.g., allylglycidyl), or vinyl esters can be used for functionalization of hpS and are currently studied. Beside the characterization of the isolates we performed various experiments to show their usability in 2D as well as 3D cell culture applications. In our 2D in vitro assays, the cell network characteristics highly depended on the numbers of cells seeded, but not on different placenta (weighing each approximately 500 g). In all experiments performed, a significantly higher network complexity was observed using hpS coatings (p<0.001) when compared to Matrigel coatings. For instance, the mean tube length using hpS coatings form close-mesh networks (e.g., like in a retina), whereas the mean tube length using Matrigel from tumor-materials rather form wide-mesh networks. The interconnected cell networks on hpS remained for around five days in vitro, even when only using minimally essential RPMI medium, whereas the cell networks on Matrigel develop faster, but also degrade faster, as reported in literature. There were no significant differences of the cell network characteristics observed on both hpS substrates, although the total protein content in Tris-NaCl is around 25% lower than Tris-urea, and it contains a different protein composition. The physiological relevance of Matrigel as a cell culture substrate is often called into question, as assays performed on Matrigel may result in false positive and false negative research results. For instance, in vitro, endothelial, but also many non-endothelial cells types such as NIH3T3-fibroblasts, melanoma, glioblastoma, breast cancer or aortic smooth muscle cell lines are already reported to form interconnected networks when seeded on Matrigel. Therefore, we performed an experiment using gfpNIH3T3 fibroblasts. While these cells did not form networks on hpS, they spontaneously formed networks on Matrigel within the first 24 h, which again confirms that Matrigel can also provoke false positive or negative research results. Using a physiological Tris 0.15 M NaCl buffer to precipitate hpS would substitute the remaining dialysis steps, however the protein concentration and the final in vitro bioactivity was low when compared to our used Tris 0.5 M NaCl precipitation buffer. We also showed that hpS can also be used as a coating material or a cell culture medium supplement using HaCaT keratinocytes, HepG2 and primary hepatocytes, NIH3T3 fibroblasts, PC-12, hAMSCs, ASCs, and other cell types. However, more studies are currently studied to assess its full potential as a coating material or as a medium supplement. After the 2D experiments we translated our findings to 3D approaches since they are known to mimic the in vivo situation more accurate, when compared to 2D in vitro techniques. Indeed, many new technologies have been explored over the last years to pattern vascular cells in 3D hydrogels, and to guide vascular organization via chemical or mechanical signals. In addition, various publications have shown that channeled hydrogels improve the vascularization rate in 3D matrices. In order to create a hierarchical channeled blood vessel network, various fabrication techniques have already been utilized to create channel networks in hydrogels including (1) removable structures, (2) 3D laser-assisted printing of photo-hydrogels or (3) planar processing such as layer-by-layer UV radiation and polymerization of hydrogels.

For our experiments, we mixed hpS with various natural proteins to form 3D hydrogels, to provide a useful material for many in vitro applications such as 3D cell culture, bio printing or perfused constructs.

For instance, in our 3D vasculogenesis studies, freeze-dried human fibrinogen, a clinically established product for decades, was mixed with hpS Tris-NaCl to induce a randomly-oriented vasculogenic cell network in 3D after around 10 days of in vitro culture, where as in traditional fibrin clots mixed with thrombin, no vasculogenic effects were observed within this time frame. In other experiences, human primary colon organoids were cultivated in a hpS/fibrin clot in the same manner as in Matrigel, which could be used for potential clinical applications.

Depending on the intended use the composition of the present disclosure may further comprise one or more compounds selected from the group consisting of antimicrobial agents, analgesic agents, local anesthetic agents, anti-inflammatory agents, immunosuppressant agents, anti-allergenic agents, enzyme cofactors, essential nutrients, growth factors, human thrombin cytokines, and chemokines, or combinations thereof.

The present disclosure also provides a composition further comprising natural polymers or synthetic polymers.

The present disclosure describes a process for obtaining fully-human biomolecules derived from the human placenta. The approach uses directed fractionation and separations techniques to derive a complex of active human biomolecules isolated from the human placenta. Specifically, the extract is obtained by a Tris-NaCl buffer extraction.

The present disclosure describes a process for preparing a biologically active human substrate comprising the steps of providing a sample from human placenta; removing blood from said sample to obtain a crude extract; solubilizing proteins in said crude extract using a 0.5 M Tris-NaCl buffer; separating solid materials from the solubilized protein extract mixture; optionally dialyzing the solubilized protein extract; and obtaining the biologically active human placenta substrate.

In specific embodiment of the invention the extraction step is carried out by using at least 0.2, 0.3, 0.4, most preferably 0.5 M, 0.6, 0.7, 0.8, 0.9, 1, 2, 3, 4, 5, or 6 M Tris-NaCl buffer. In one embodiment a Tris 0.5 M NaCl buffer is used.

In order to avoid toxic denaturizing detergents, the extraction step is carried out in the absence of urea, guanidine-HCl, sodium dodecyl sulfate (SDS), Triton X-100 or enzymatic digestives such as e.g. pepsin.

The herein disclosed composition is suitable for a variety of applications. Musculoskeletal disorders account for more than 50% of the harmful disabilities reported by adults and require the regeneration of muscles, tendons, ligaments, joints, peripheral nerves and supporting blood vessels.

The treatment of burns and chronic wounds requires a rapid response, wherein in most cases skin grafting is required. Regenerative medicine helps to reduce the aftereffects of the general treatments used in burns, including the reduction of scars and skin infections. Complications of wound healing are an increasing threat to patients, public health and the economy. Over 300 million people are currently suffering from chronic or non-healing wounds. The success of many current therapies in regenerative medicine requires the ability to create and control stable vascular networks within tissues.

Cardiovascular diseases (CVD) encompass to a wide range of diseases such as coronary heart disease, cerebrovascular disease, peripheral artery disease, rheumatic heart disease, congenital heart disease, deep vein thrombosis and pulmonary embolism. A heart attack, known as myocardial infarction (MI), occurs when the blood supply to the heart is disrupted, causing heart cells to die from oxygen deficiency. Regenerative medical technologies may add to rescue, replace and revitalize these damaged heart tissues.

### EXAMPLES

The Examples which follow are set forth to aid in the understanding of the invention but are not intended to, and should not be construed to limit the scope of the invention in any way. Such methods are well known to those of ordinary skill in the art.

### Material and Methods

If not stated otherwise all reagents were from Sigma Aldrich and of analytical grade.

### Collection of Human Placenta Tissue

Placenta material was collected after caesarian section from the Kepler University Clinics Linz, Austria (with the consent of the local ethical board and informed consent from all donors). Tissues were stored at −20° C. up to 3 months until isolation was performed.

Human Placenta Substrate (hpS) Isolation

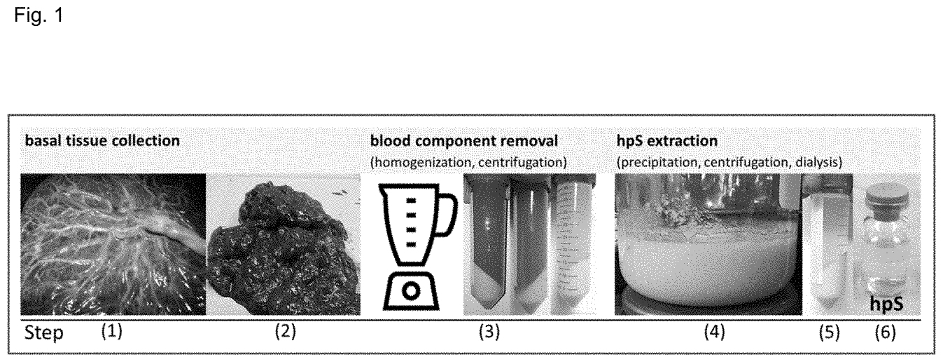

All isolation steps were performed in a cold-room at 4° C. After thawing, the amnion, chorion and umbilical cord were removed. The resulting basal villous tissue was used for the isolation process (FIG. 1). Blood components were removed by repetitive homogenization steps, where 200 g basal placenta tissue were homogenized in 400 mL of a Tris NaCl buffer (0.05 M Tris, 3.4 M NaCl, 4 mM EDTA, 2 mM N-Ethylmaleimide (NEM), pH 7.4) using a grinder (Braun Type 4184, Germany) and subsequently centrifuged at 7,000×g for 5 min using a Heraeus Multifuge (Beckman Instruments GmbH Type 1 S-R, Austria). The supernatant containing blood components was discarded and the pellets resuspended in 400 mL of fresh Tris-NaCl buffer. This procedure was repeated two additional times. For hpS extraction, 100 g of pellets were suspended in 100 mL of either a Tris-NaCl buffer (0.05 M Tris, 0.5 M NaCl, 4 mM EDTA, 2 mM N-ethylmaleimide (NEM), pH 7.4) or a Tris-urea buffer (0.05 M Tris, 2 M urea, 0.15 M NaCl, 4 mM EDTA, 2 mM N-ethylmaleimide (NEM), pH 7.4) and stirred for 24 h on a magnetic stir plate at 200 rpm at 4° C. The suspensions were centrifuged at 14,000×g for 20 min. The pellets were discarded (some pellets were kept for additional measurements; a second precipitation step) and the supernatants containing hpS were collected and dialyzed against 40× volume PBS buffer in 6-8 kDa cut-off dialysis membranes (Fisher Cellulose, #21152-5). PBS was changed 3 times. The resulting substrates (hpS Tris-NaCl; hpS Tris-urea) were stored at −80° C. Aliquots of hpS were further dialyzed against 40× volume aqua dest in 6-8 kDa cut-off dialysis membranes (Fisher Cellulose, #21152-5) to remove the remaining salts and freeze-dried and amino acid quantification was performed.

### Biochemical Characterization of hpS

### Total Protein Content

Protein content of hpS was determined using a bicinchoninic acid assay (BCA; Thermo Scientific, 23228, Vienna, Austria), according to the manufacturer's instructions. Briefly, dilutions of bovine serum albumin (BSA) were used to generate a standard curve. Samples/standards and BCA buffer were pipetted into 96-well plates (Greiner Bio-one, Kremsmunster, Austria) and incubated at 37° C. for 30 min. Then, the absorbance was measured at 562 nm using an Omega POLARstar 140 plate reader (BMG Labtech, Ortenberg, Germany).

### Papain Digestion

Papain digestion was performed as described elsewhere. Freeze-dried hpS was digested with 3 IU/mL papain from papaya latex (75 mM NaCl, 27 mM Na Citrate, 0.1 M NaH₂PO₄, 15 mM EDTA and 20 mM L-Cysteine, pH 6.0) at 60° C. for 24 h before assessing DNA and GAG content.

### DNA Content

CyQuant stain (Thermo Fisher Scientific, Vienna, Austria) was used as described by the manufacturer for DNA quantification. Briefly, papain digested samples and standards from DNA sodium salt from calf thymus were pipetted into 96-well black microplates (Brand, Wertheim, Germany). The plate was incubated in the dark for 5 min at room temperature. Then, the fluorescence intensity was measured using an Omega POLARstar 140 plate reader (BMG Labtech, Ortenberg, Germany) at 485 nm with a reference wavelength of 520 nm.

### Glycosaminoglycan Quantification

Dimethylmethylene Blue (DMB) was used for GAG quantification. Papain digested samples were diluted in aqua dest before measurement and 100 μL of standard/samples were pipetted into 96-well plates (Greiner flat bottom, Kremsmunster, Austria). 200 μL of DMB color solution (46 μM DMB, 40 mM NaCl, 40 mM Glycine in dH₂O, pH 3) were added and optical absorbance was immediately measured at 530 nm with a reference wavelength of 590 nm using an Omega POLARstar 140 plate reader (BMG Labtech, Ortenberg, Germany).

### SDS PAGE Gel Electrophoresis/Western Blot

SDS PAGE and Western blot analysis was performed using the XCell SureLock™ Mini-Cell Electrophoresis System (Invitrogen, Vienna, Austria). 20 μg of sample proteins per lane was resolved on 3-8% gels and a marker (Gel filtration standard 151-1901, BioRad, Vienna, Austria) and 12% gels and a marker (Protein marker V, VWR, Vienna, Austria). The gels were stained with Coomassie Brilliant Blue R-250 (BioRad, Vienna, Austria), or transferred onto nitrocellulose membranes for Western blot analysis (Peqlab, Germany). The membranes were blocked with 5% milk in TBS buffer containing 0.1% Tween (TBS/T), and primary antibodies against collagen-1 (AB 34710, Abcam, Cambridge, USA), collagen-4 (AB6586, Abcam, Cambridge, USA) or laminin-111 (AB11575, Abcam, Cambridge, USA), in 5% BSA-TBS/T were incubated at 4° C. overnight. Membranes were further incubated in 5% milk-TBS/T for 1 h containing secondary antibodies (LI-COR Biosciences, Lincoln, USA) and the signals were detected using the Odyssey Fc infrared imaging system (LI-COR Biosciences, Lincoln, USA).

### Angiogenesis Array

Relative levels of angiogenesis-related proteins from hpS Tris-urea or Tris-NaCl were determined using human angiogenesis antibody Arrays C1000 (RayBio, USA) according to the manufacturer's instructions. Membranes containing 43 different cytokine antibodies (duplicates) were blocked and incubated with 1 mL of 3 pooled, normalized hpS samples o/n at 4° C. All residual steps were performed at room temperature. After washing, biotinylated antibody incubation for two hours and a second wash, the membranes were incubated with HRP streptavidin for two hours, washed and chemiluminescence was detected using myECL Imager (Thermo Scientific, USA).

Data analysis was performed according to the manufacturer's instructions. Each membrane was exposed to obtain high signal-to-noise ratios using the gel documentation system (myECL Imager, Thermo Scientific, USA). The spot signal intensities were further analyzed using mylmage Analysis Software Version 1.0 (Thermo Scientific, USA). One array was defined as “Reference Array”, to which the other arrays were normalized to and a working template was created. For each spot, the signal density (intensity/area) was used for numerical data transformation. The background signal was subtracted from raw numerical densitometry data and normalized to the positive control signals—standardized amounts of biotinylated IgG.

### VEGF ELISA

A human VEGF ELISA was used as described by the manufacturer (R&D Systems, Catalog #DY990). Briefly, the antibody was diluted in PBS and coated on 96-well plates overnight (100 μL per well). The wells were washed three times with a buffer containing 0.05% Tween® 20 in PBS. Then, the plates were blocked with 300 μL PBS containing 1% BSA for 1 h and washed again trice. 100 μL of sample or standards were added and the plates were incubated for 2 hours, and then washed again trice. A secondary antibody was added and the plates were incubated for 2 hours and washed again. Finally, 100 μL of streptavidin conjugated to horseradish peroxidase was added per well for 20 minutes and the optical density was assessed using an Omega POLARstar 140 plate reader (BMG Labtech, Germany) at 450 nm.

### Chromogenic Thrombin Assessment

Human thrombin was assessed using chromogenic measurements (Technothrombin® TRA, Technoclone, Vienna, Austria) according to the manufacturer's instructions. Briefly, the detergents were diluted in aqua dest and pipetted in black NUNC 96 well plates and calibration curves were measured at 37° C. using a fluorometer (BMG Labtech, Ortenberg, Germany) at 360 nm/460 nm extinction/emission for 10 min in 30 s measurement intervals, before the analysis of hpS Tris-NaCl was assessed for 60 min in 1 min measurement intervals. All plate readings were immediately performed after pipetting the samples/substrate.

### Characterization of Antimicrobial Effects of hpS Tris-NaCl

hpS Tris-NaCl from three different isolations was pooled and UV sterilized in 6-well plates for 30 min. Aliquots were stored at −20° C. until further use. The bacteria strains (Table 2) were grown in lysogeny broth (LB medium; LB Broth, Molecular Genetics Granular, Miller) an without antibiotics. Then, the cultures were diluted 1:6 to 1:10 with fresh medium and grown for 30 min with shaking (200 rpm) at 37° C. to exponential growth phase (OD600 0.5-0.7). Based on the OD600 measurement the bacteria concentrations were calculated according to the formulas given in table 2 and the suspension was diluted to 2×10⁶ bacteria/mL. 50 μL of these dilutions (1×10⁵ bacteria) were mixed with 50 μL of hpS Tris-NaCl in a flat-bottom 96-well plate. OD600 values were measured with an Omega POLARstar 140 plate reader (BMG Labtech, Ortenberg) for a total time of 7 h. For the negative controls, hpS Tris-NaCl was replaced by PBS. Each sample was measured in triplicates and the experiment was performed three times for statistical analysis.

TABLE 2
Bacteria strains used

| | | Dilution | |
|---|---|---|---|
| Strain | Supplier | factor | Formula |
| Escherichhia coli | ThermoFisher | 1:6 | OD₆₀₀ = 1.0 ≙ |
| TOP10 | (C404010) | | 8 · 10⁸ bacteria/mL |
| Escherichhia coli | ATCC (700926) | 1:8 | OD₆₀₀ = 1.0 ≙ |
| MG1655 | | | 4 · 10⁸ bacteria/mL |
| Staphylococcus | Dr. Plätzer, | 1:10 | OD₆₀₀ = 1.0 ≙ |
| carnosus | University of | | 8 · 10⁷ bacteria/mL |
| | Salzburg | | |
| Staphylococcus | Dr. Plätzer, | 1:6 | OD₆₀₀ = 1.0 ≙ |
| capitis | University of | | 1.6 · 10⁷ bacteria/mL |
| | Salzburg | | |

### Amino Acid Analysis

Amino acid quantification was performed using three hpS samples from three independent donors.

### Sample Preparation

Freeze-dried hpS Tris-NaCl was digested following a two-step protocol; first enzymatically and then chemically. Briefly, 75 mg of lyophilized sample were incubated with 1 mL of 0.0125% protease from Streptomyces griseus in 1.2% Iris/0.5% sodium dodecyl sulfate pH 7.5 (adjusted with 0.1% HCl) solution for 72 h at 37° C. Then 1 mL of 4% formic acid in ddH₂O was added for chemical pre-digestion and the suspension was incubated for 2 h at 108° C. followed by lyophilization. The dried samples were then incubated for 2 h with 5 mL of a solution containing 0.6% TRIS and 7 M guanidinium hydrochloride pH 8. After centrifugation (Sigma centrifuge, 3-18 K) of the sample at 4,800 rpm for 15 min at 4° C., 1 mL of the supernatant was combined with 0.5 mL 4 M methanesulfonic acid solution containing 0.2% tryptamine and was incubated for 1 h at 160° C. Subsequently, the solution was quantitatively transferred into a 5 mL volumetric flask, 225 μL 8 M NaOH and 0.25 mL internal standard were added and the flask was filled up with 2.2 M sodium acetate solution. The samples could then directly be used for HPLC analysis.

### HPLC Standard Preparation

A multi-amino acid standard mix was prepared by mixing the amino acid standard, a solution containing 2.5 mM each of asparagine, glutamine and tryptophan in MQ, a solution containing 2.5 mM each of taurine and hydroxyproline in 0.1 M HCl and a solution of the internal standards, i.e. 25 mM each of norvaline and sarcosine in 0.1 M HCl. Ten different concentrations of this standard mixture, ranging between 45 mg/L and 0.5 mg/L, were used for calibration.

### HPLC Analysis

The HPLC system Ultimate 3000 (Thermo Fisher Scientific, USA) was equipped with a pump (LPG-3400SD), a split-loop autosampler (WPS-3000 SplitLoop), a column oven (Col.Comp. TCC-3000SD) and a fluorescence detector (FLD-3400RS). Chromeleon 7.2 was used for the control of the device as well as for the quantification of the peak areas. Chromatographic separation was achieved with a reversed phase column (Agilent Eclipse AAA, 3×150 mm, 3.5 μm) a guard column (Agilent Eclipse AAA, 4.6×12.5 mm, 5 μm) and a gradient using eluent (A) 40 mM NaH₂PO₄ monohydrate pH 7.8 and eluent (B) MeOH/ACN/MQ (45/45/10, v/v/v). The protocol was run with a flowrate of 1.2 mL min-1, the column oven temperature was set to 40° C. and the injection volume was 10 μL. As most amino acids have no fluorophore in their structure, an in-needle derivatization step was performed using 0.4 M borate buffer, 5 mg/mL ortho-phthaldialdehyde (OPA) in 0.4 M borate buffer containing 1% of 3-MPA, 2.5 mg/mL FMOC and 1 M acetic acid for pH adjustment. In order to guarantee sample quantification despite the derivatization step, every sample was spiked with 25 mM sarcosine in 0.1 M HCl and 25 mM norvaline in 0.1 M HCl as internal standards. Primary amines and Norvaline were detected at Ex 340 nm/Em 450 nm and secondary amines and Sarcosine were detected at Ex 266 nm/Em 305 nm.

### 3D Solidification of hpS Tris NaCl

Collagen-1/3 (COL1/3): Freeze-dried COL1/3 from human placenta was resolved in PBS buffer to a concentration of 8 mg/mL, hpS was added (1+1 vol.) and the final solution was incubated at 37° C. to achieve solidification.

Gelatin: Gelatin (Merck, 4078) was diluted in hpS at room temperature to a final concentration of 3% and the solution was incubated at 4° C. to achieve solidification.
Fibrinogen: Fibrinogen (Tisseel, Baxter, Austria) was diluted in EGM-2 medium to a concentration of 10 mg/mL, only hpS was added (1+1 vol.) and the final solution was incubated at 37° C. to achieve solidification.
Agarose: Agarose (Biozym LE Agarose, Oldendorf, Germany) was resolved in aqua dest to a concentration of 2% at 175° C. until the suspension became clear. After cooling to 40° C., hpS was added (1+1 vol.) and the solution was incubated at 4° C. to achieve solidification.
Agar-agar: Agar-agar (Fluka, St. Louis, USA) was resolved in aqua dest. to a concentration of 3% at 90° C. and after cooling to 40° C., hpS was added (1+1 vol.) and the solution was incubated at 4° C. to achieve solidification.

### 2D in Vitro Bioactivity

### HUVEC Isolation

Human umbilical vein endothelial cells (HUVECs) were isolated from three donors. HUVECs were isolated from biological materials obtained from healthy donors with the authorization of the local ethics committee of upper Austria and informed consent by the donors. Cells (p5-p9) were cultured in EGM-2 (Lonza), supplemented with 5% FCS. Isolated HUVEC were retrovirally infected with expression vectors for fluorescent proteins using the Phoenix Ampho system.

### HUVEC Seeding Density

Vasculogenesis assays were performed as described. Briefly, 50 μL of hpS Tris-NaCl or hpS Tris-urea extracted from the same tissue were pipetted in 96 well plates, UV sterilized for 30 min and incubated at 37° C. for 3 h. Thereafter, different cell numbers ranging between 5,000 and 25,000 HUVEC from the same donor (passage 8) were seeded on hpS in 100 μL of EGM-2 medium (Lonza, Basel, Switzerland).

After two days of cultivation, the formed cell networks were imaged and analyzed. Fluorescence microscopic pictures were taken from two different fields per well with a Leica epifluorescence microscope DMI6000B (Vienna, Austria) and processed in a blinded way using Adobe Photoshop software (Adobe Systems, San Jose, USA) by adjusting contrast and brightness. Then, tube formation was analyzed using AngioSys 2.0 software (TCS Cellworks, London, UK) and the AngioSys values were statistically analyzed using Prism 5 (Graphpad).

### Immunohistochemistry

Formed HUVEC networks on hpS Tris-NaCl were stained with anti-CD 31 and vascular endothelial cadherin (VeCad) antibodies (BD Pharmigen, San Diego, USA) after two days of cultivation. The medium was aspirated and cells were washed with PBS before fixation in 4% formaldehyde for 10 min and washing with PBS for 5 min. All following steps were performed in the dark. Cells were incubated in PBS containing 1% BSA and CD31 antibody (BD Pharmigen, 555445) mouse α-human 1:100; or VeCad antibodies (BD Pharmigen 560411) mouse α-hum 1:100 for 30 min at room temperature. Then the cells were washed twice with PBS for 5 min and the secondary antibody AK Alexa Fluor 488 goat a mouse IgG (Life Technologies a11029, 1:100) in PBS containing 1% BSA was added and incubated for 30 min at room temperature. Plates were washed twice with PBS for 5 min and DAPI was added (1:1,000). After a final PBS washing step, the networks were imaged.

### Comparison of Substrates

To determine the influence of substrates on the cell networks, 50 μL of Matrigel, hpS Tris-NaCl or Tris-urea from the same tissue were pipetted in 96 well plates, UV sterilized and incubated at 37° C. for 3 h. 20,000 gfpHUVEC from a donor (p8) were seeded in 100 μL of EGM-2 medium (Lonza). After 3 h, the medium was replaced with 100 μL of minimal essential RPMI-1640. Medium change was performed every second day and the networks were analyzed after 6/24/48/72/96/120 h.

### Single Placenta Tissue Comparison

To determine the consistency of the isolation method using single tissues, hpS Tris-NaCl was isolated from 3 different tissues, each weighing around 500 g. 50 μL of Matrigel or hpS were pipetted in 96 well plates, UV sterilized and incubated at 37° C. for 3 h. 20,000 gfpHUVEC from a donor (p7) were seeded in 100 μL of EGM-2 medium (Lonza). After 3 h, the medium was replaced with 100 μL of minimal essential RPMI-1640 medium and the networks were analyzed every 24 h.

gfpN/H3T3 Fibroblast Cultivation

NIH3T3 mouse fibroblasts were purchased from DSMZ (No: ACC59, Braunschweig, Germany) and cultured in DMEM high glucose supplemented with 10% FCS and 1% glutamine. 50 μL of Matrigel or hpS Tris-NaCl were pipetted in 96 well plates, UV sterilized for 30 min and incubated at 37° C. for 3 h. Then, 20,000 gfpNIH3T3 fibroblasts were seeded on coated or uncoated wells (control) in 150 μL of DMEM medium and after 24 h, the cells were analyzed.

### HUVEC Cell Culture Supplementation

To determine the potential of hpS Tris-NaCl as a cell culture medium supplement, 20,000 gfpHUVEC from a donor (p7) were seeded in 150 μL of EGM-2 medium (Lonza) or EGM-2 medium supplemented with 30% of UV sterilized hpS in uncoated 96 well plates, or in 150 μL of EGM-2 medium on hpS 0.5 M Tris-NaCl coated plates or on a Tris 0.15 M NaCl extracted substrate. The networks were analyzed after 24 h.

### hpS to Compensate FCS

FCS substitution experiments were performed with HaCaT, HepG2, NIH3T3 fibroblasts, or hAMSC, as examples. For instance, 5,000 HaCaT cells were cultivated 24 well plates. Viability rates were assessed using MTT tests and morphologic changes were microscopically analyzed. HepG2 cells were cultivated in 500 μL of DMEM high glucose, supplemented with 10% FCS or 10% hpS, 1% glutamine and 1% antibiotics (AntiAnti®) in 48 well plates. Viability rates were assessed using MTT tests and morphologic changes were microscopically analyzed.

### hpS as 2D Coating Material

Coating experiments were performed with NIH3T3 fibroblasts, hepatocytes, or PC-12 cells, as examples. For instance, HIH3T3 fibroblasts were cultivated in 500 μL of DMEM high glucose, supplemented with 10% FCS and 1% glutamine, on either hpS- or Matrigel-coated wells in 3 different coating concentrations (1.5 mg/mL, 150 μg/mL or 15 μg/mL). Viability rates were assessed using MTT tests and morphologic changes were microscopically analyzed. Primary rat hepatocytes were cultivated in 500 μL of DMEM high glucose, supplemented with 10% FCS and 1% glutamine, on either hpS- or Matrigel-coated wells (100 μg/mL). Four hours after cell seeding, Easy4You viability assays were assessed according to the manufacturer's instructions. PC-12 cell lines were purchased from ECACC (#88022401, Salisbury, U.K.) and cultured in DMEM high glucose supplemented with 15% FCS, 1% glutamine and 1% Penstrep. 24 well plates were incubated with 250 μL of Matrigel, collagen-1 or hpS at 100 μg/mL and UV sterilized for 30 min. Coating solutions were removed and 12,000 cells were seeded (6000 cells/cm2, n=12) on the coated wells and incubated at 37° C. for 2 h. Photographs were taken after 2 days using an epifluorescence microscope (DM16000B, Leica GmbH, Vienna, Austria) and the outgrowth was analyzed as described. Briefly, microscopy pictures were processed in a blinded manner with Adobe Photoshop software by adjusting contrast/brightness. Then the neurite outgrowth was analyzed using AngioSys software (TCS Cellworks, London, UK). The obtained values were further statistically analyzed using Prism 5 (Graphpad, Calif., USA).

### 3D in Vitro Bioactivity

Mixing hpS Tris-NaCl with fibrinogen for 3D studies

hpS Tris-NaCl was pipetted in 6 well plates and the wells were UV sterilized for 30 min. Meanwhile, fibrinogen (Tisseel, Baxter) was diluted in EGM-2 medium to a concentration of 20 mg/mL at 37° C. 500 μL of this suspension was mixed 1:1 vol. with 500 μL EGM-2 medium containing 500.000 gfpHUVEC. This suspension was further mixed (1:1 vol.) with hpS or 0.4 U thrombin (Tisseel, Baxter) as sample control and incubated at 37° C. for 2 h. After polymerization, 3 mL of EGM-2 medium were added. Medium was changed every third day and the wells were analyzed after 11 days of cultivation.

### Scanning Electron Microscopy (SEM)

For SEM analysis of the fibrin gels, they were fixed in 4% formaldehyde followed by sample dehydration using graded ethanol concentration series and hexamethyldisilazane. Samples were sputter-coated with Pd—Au using a Polaron SC7620 sputter coater (Quorum Technologies Ltd, UK), and examined at 15 kV using a JEOL JSM-6510 scanning electron microscope (Jeol GmbH, Japan).

### Organoid Culture

Fibrin (Baxter) was diluted in cell culture medium to a concentration of 20 mg/mL while primary malignant colon tumor cells were harvested and added to this suspension (10 mg/mL fibrinogen and 2,000 cells/μL medium), or to Matrigel (control). Thrombin (Baxter) was diluted in hpS to a concentration of 0.8 U/mL and 1:1 vol. mixed with the cell/fibrinogen suspension to a final concentration of 5 mg/mL fibrinogen, containing 1,000 cells/μL and 0.4 U thrombin. 100 μL of this suspension or Matrigel are added per well in a 24 well plate and the plate was incubated at 37° C. for 30 minutes, to clot. Then, 50 mL of media were added to each well and microscopic images are obtained daily.

### Statistical Analysis

All experimental data is presented as mean±standard deviation (SD) and P-values <0.05 were considered statistically significant. Normal distribution of data was tested with the Kolmogorov-Smirnov Test. All calculations were performed using GraphPad Prism version 6.00 (GraphPad software, San Diego, Calif., USA).

### Results

Extraction of Human Placenta Substrate (hpS)

A flow chart of the isolation method is depicted in FIG. 1. In average, around 300-350 mL of hpS was extracted from single placenta tissues, each weighing around 500 g.

Compositional assessment of hpS

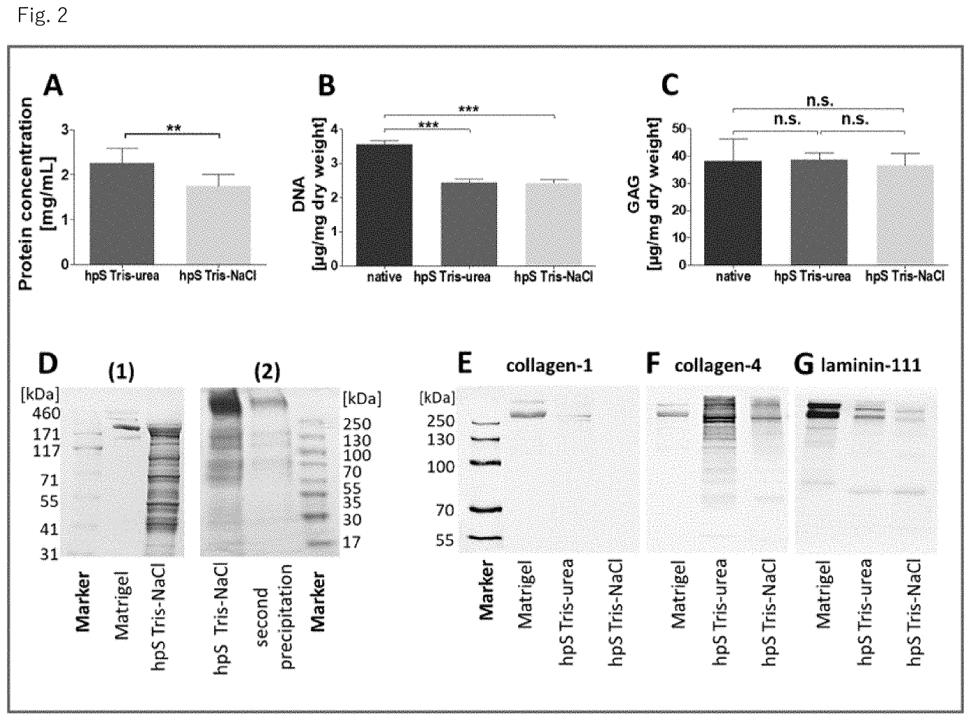

To assess the total protein concentration of both substrates, BCA assay was performed (FIG. 2A). The protein concentration of hpS Tris-NaCl (1.74±0.26 mg/mL) was significantly lower when compared to hpS Tris urea (2.26±0.32 mg/mL).

In order to assess the DNA content in hpS, CyQuant stain was used (FIG. 2B). The mean DNA content of both hpS was significantly lower compared to native placenta tissue (3.56±0.10 μg/mg dry weight), but no significant difference between hpS Tris-urea and hpS Tris-NaCl was detected (hpS Tris-urea 2.42±0.05 and hpS Tris-NaCl 2.41±0.02 μg/mg dry weight).

DMB assays were performed to determine the GAG content within hpS (FIG. 2C). There was no significant difference among native placenta, hpS Tris-urea and hpS Tris-NaCl (38.21±6.64, 38.74±2.12 and 36.4±4.04 μg/mg dry weight), respectively.

SDS-PAGE was performed to visualize the composition of proteins in hpS Tris-NaCl (FIG. 2D(1)). hpS Tris-NaCl shows various protein bands ranging from 30 kDa up to around 500 kDa (19±5), whereas Matrigel consisted of significantly fewer protein bands (3±1). A second use of the pellet (after the first Tris-NaCl precipitation) for an additionally second Tris-NaCl precipitation step yielded lower protein concentrations (FIG. 2D(2)). Western blot analysis shows, collagen-1 was present in hpS Tris-urea and Matrigel, but not in hpS Tris-NaCl, whereas collagen-4 and laminin-111 were detected in both hpS substrates (FIG. 2E-G).

An antibody-based angiogenesis array was used to assess the angiogenic profile of hpS (FIG. 3). There were higher levels of in total 43 different proteolytic enzymes, immune related cytokines, growth factors and angiogenic chemokines assessed in hpS Tris-NaCl when compared to hpS Tris-urea. Angiogenin, a potent stimulator of angiogenesis, was the most prevalent angiogenic chemokine in both hpS substrates. Other chemokines including angiostatin (ANG), growth related oncogene (GRO), angiopoietin or tissue inhibitors of metalloproteinases (TIMPs), proteolytic enzymes (MMP-1, MMP-9), interleukins (IL-1 β) or cytokines related to wound healing and tissue regeneration (TGF-ß1, bFGF, EGF, PDGF, IGF-1) were also detected.

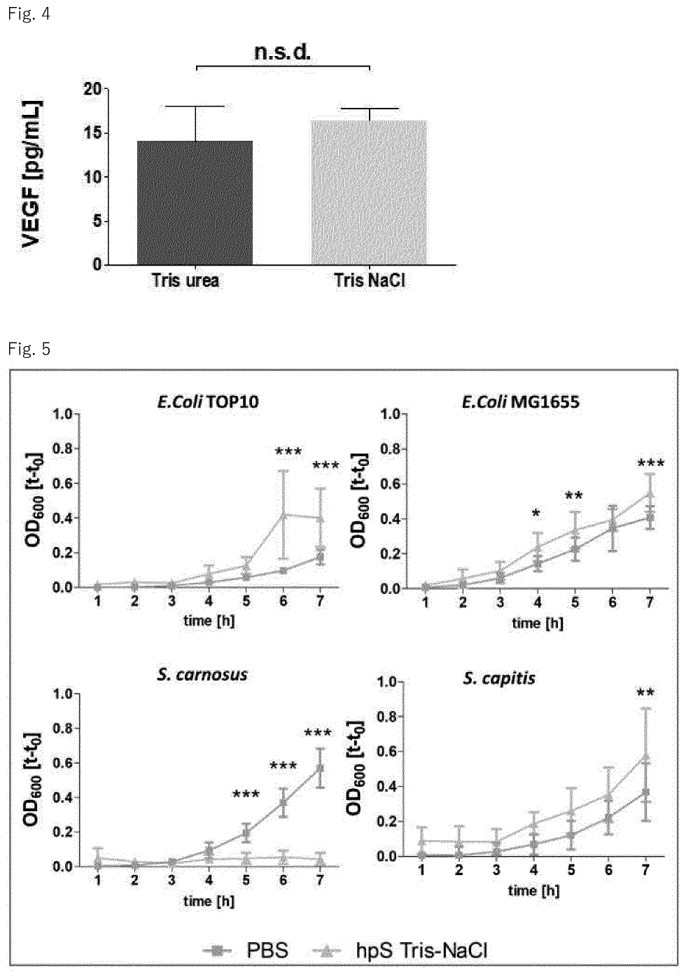

In order to assess the VEGF concentration in hpS, ELISA analysis was used (FIG. 4). There was no significant difference between Tris-urea and Tris-NaCl extracted substrates (Tris-urea 13.99±3.34, Tris-NaCl 16.28±1.25 pg/mg dry weight).

A chromogenic assay was performed to assess the presence of active thrombin in hpS Tris-NaCl. In average, 0.63±0.16 U thrombin per mL was detected in hpS Tris-NaCl.

Antimicrobial effects of hpS Tris-NaCl were tested in two gram-negative strains (E. coli TOP10, E. coli MG1655) and two gram-positive strains (S. carnosus, S. capitis). In S. carnosus, hpS Tris-NaCl showed distinct antibacterial properties and significantly delayed bacterial growth over 7 h. However, in the other strains, hpS showed a positive effect on bacterial growth (FIG. 5).

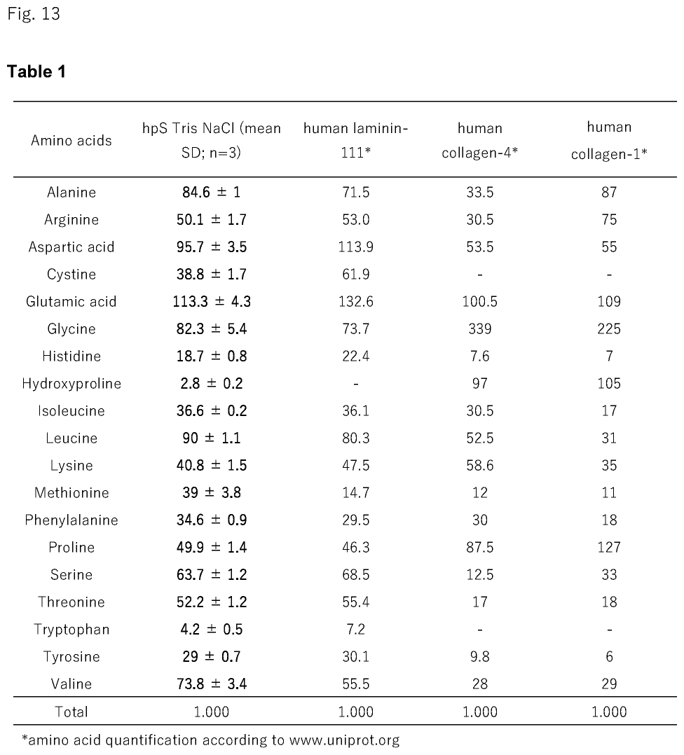

Table 1 (FIG. 13) lists the amino acid composition of hpS Tris-NaCl from three different placentas showing high amounts of glutamic-/aspartic acid, and leucine (each around 10%) and similar pattern to laminin-111.

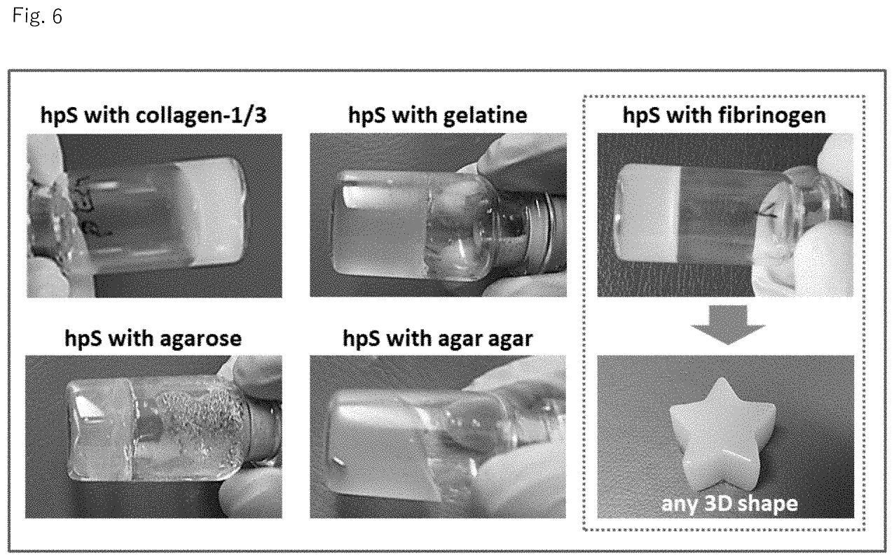

A broad variety of natural polymers, that were already used for bio printing in literature, were mixed with hpS Tris-NaCl to achieve a stable 3D solidification at 4° C. or 37° C. (FIG. 6).

### 2D Biocompatibllity of hpS

### HUVEC Seeding Density

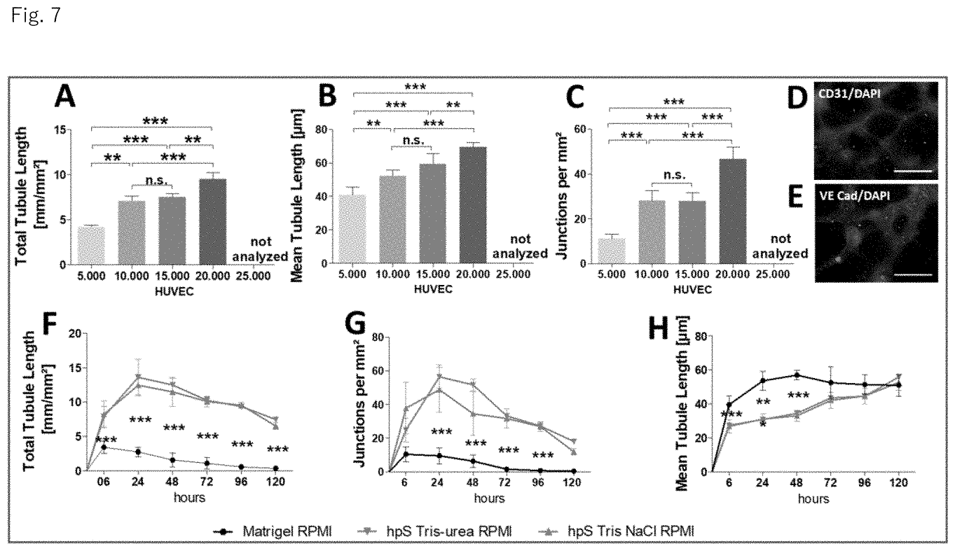

Different cell numbers (5,000-25,000 cells/well) were cultured for two days on hpS Tris-NaCl or hpS Tris-urea and the networks were analyzed (FIG. 7A-C). At seeding densities from 10,000 to 20,000 cells in 96 wells (30,000-60,000 cells/cm²), interconnected networks were formed in a cell number dependent manner in the first 24 h of culture. 5,000 cells developed only partial cell networks and 25,000 cells yielded confluent non-polarized cell layers that were not further analyzed. The network characteristics (total/mean tubule length, junctions) using 20,000 cells were significantly increased compared to all other cell seeding concentrations on both substrates, Tris-NaCl and Tris-urea.

### Immunohistochemistry

CD31 and vascular endothelial cadherin (VeCad), both marker for endothelial cells, were detected on HUVEC that assembled into an interconnected cell network (vasculogenesis) when seeded on hpS Tris-NaCl (FIG. 7D,E).

### Comparison of Substrates

20,000 gfpHUVEC from the same donor were seeded on hpS Tris-NaCl, hpS Tris-urea or Matrigel, and the cells were cultivated using minimal essential RPMI medium. The networks were analyzed after 6/24/48/72/96 and 120 h. On Matrigel, the network characteristics (total/mean tube length, number of tubules/junctions) were significantly lower when compared to both hpS. There were no significant differences in cell network characteristics between hpS Tris-NaCl and Tris-urea from the same donor using RPMI medium (FIG. 7F-H).

### Single Placenta Tissue Comparison

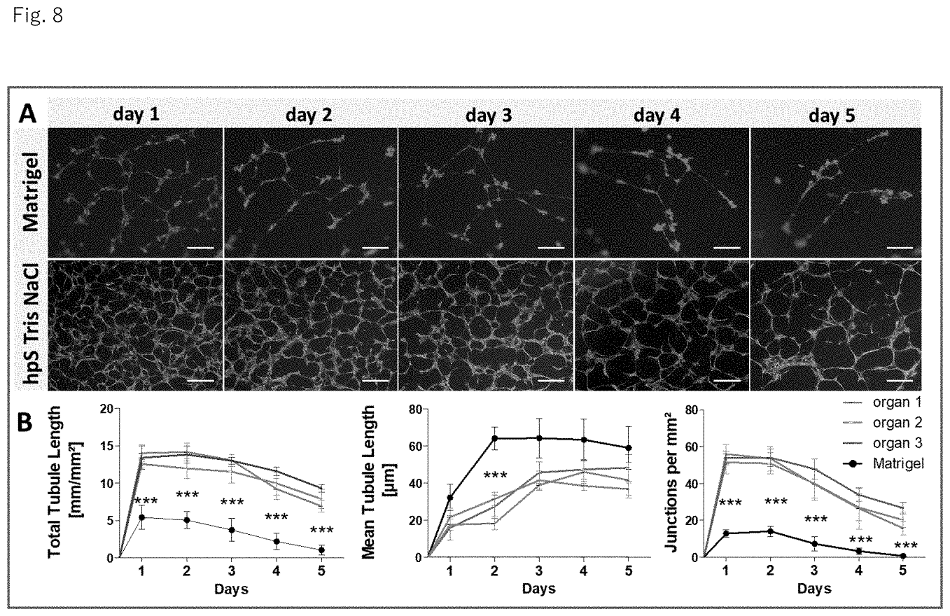

Representative images of formed networks after two days are shown in FIG. 8A. There was no significant difference observed in the network characteristics (total/mean tube length, number of tubules/junctions) between 3 different placentas, each weighing around 500 g (FIG. 8B), but the network characteristics were significantly increased when compared to Matrigel.

gfpN/H3T3 Fibroblast Cultivation

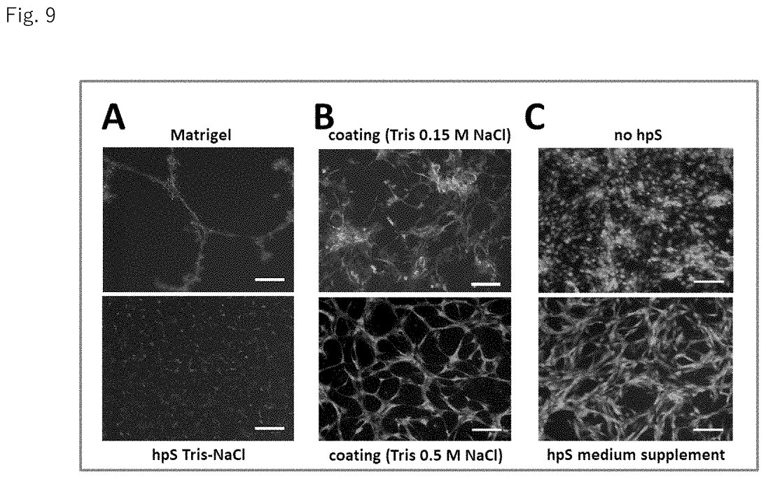

Fibroblasts spontaneously formed networks when seeded on tumor-derived Matrigel, but not on hpS Tris-NaCl (FIG. 9A). Substrates from human placenta extracted with a Tris 0.15 M NaCl buffer (physiologic) showed a different cell morphology and a lower in vitro performance when compared to hpS Tris 0.5 M NaCl (FIG. 9B). HUVEC polarization was also observed by applying hpS Tris-NaCl as a cell culture medium supplement without further hpS coatings (FIG. 9C)

### hpS to Substitute FCS

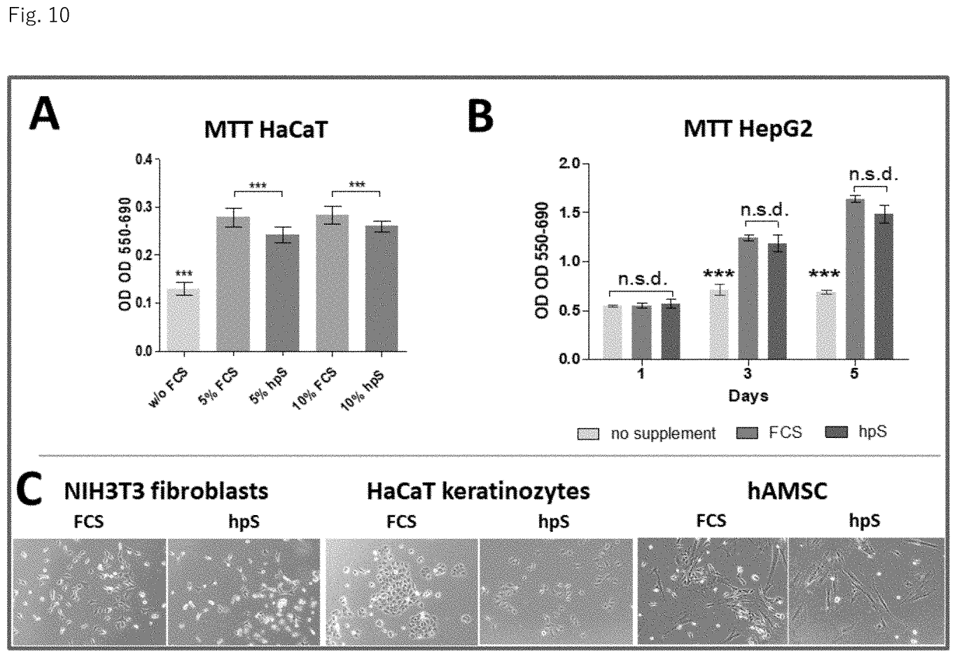

HaCaT cells were successfully cultivated in cell culture medium supplemented with 5% or 10% hpS instead of FCS (FIG. 10). Although viability using 5% hpS and 10% hpS was lower than in FCS-supplemented culture media it still was significantly higher than in the control group without supplement. When using 5% hpS, the viability rates were 86% when compared to 5% FCS. When using 10% hpS, the viability rates were 91% when compared to 10% FCS. Without any supplementation, the viability rate was 45.5% when compared to FCS supplemented cell culture medias (FIG. 10A). HepG2 cells were successfully cultivated in cell culture medium ether supplemented with 10% hpS or 10% FCS with no significant difference, but significant higher viability rates when compared to the control group without supplement (FIG. 10B). Various other cell types were cultivated using hpS instead of FCS supplemented medium (FIG. 10C).

### hpS as 2D Coating Material

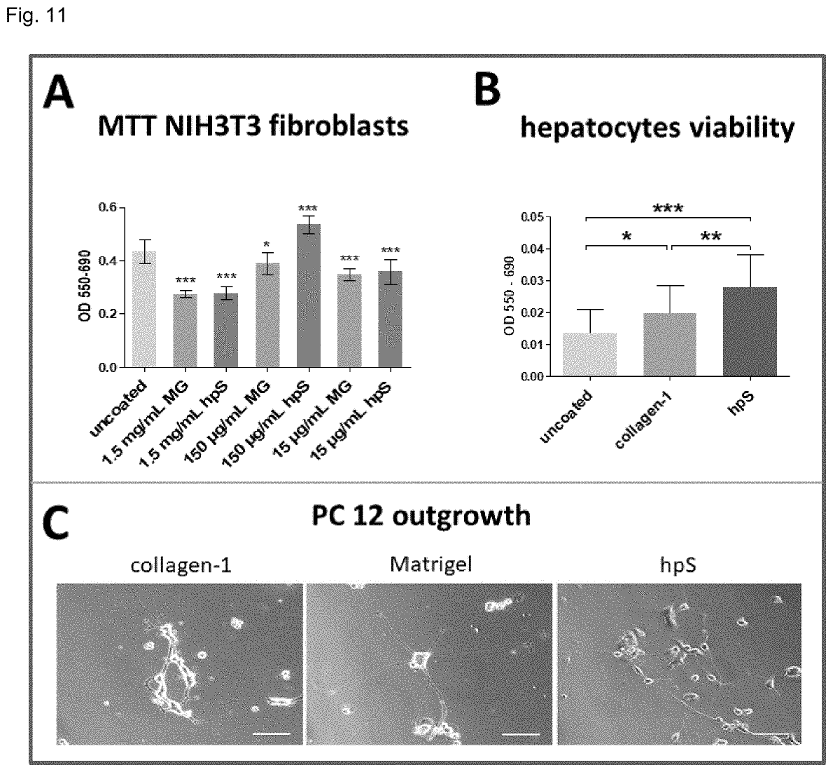

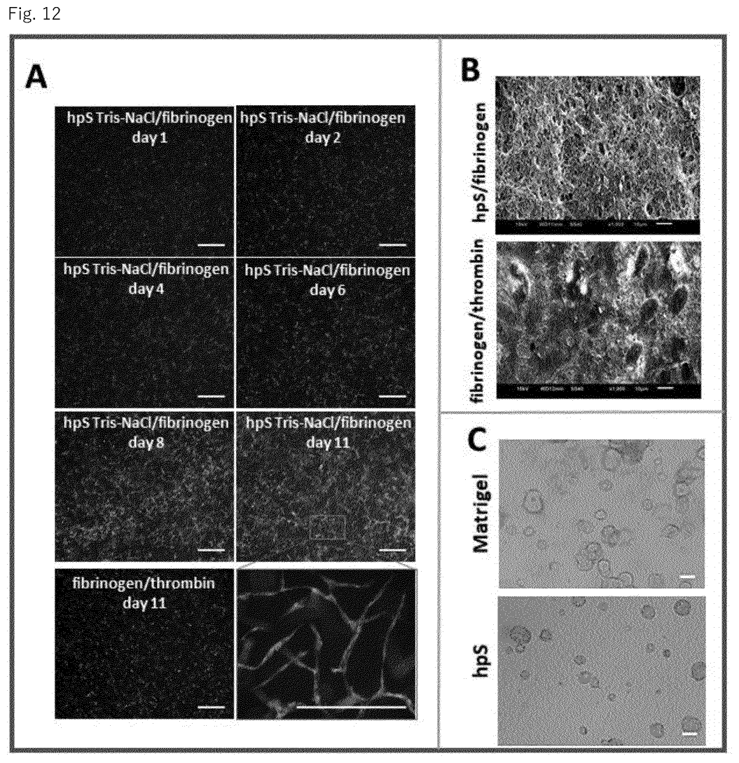

hpS was well suited as coating material (FIG. 11). Using NIH3T3 fibroblasts, the viability rates were significantly higher using hpS at 150 μg/mL, when compared to Matrigel or other coating concentrations (FIG. 11A). Using primary rat hepatocytes, the viability rates were significantly higher using hpS when compared to collagen-1 coatings four hours after seeding (FIG. 11B). An outgrowth assay was used to analyze PC 12 cells on hpS coated wells and compared with Matrigel or collagen-1 coated wells. After 2 days, the total neurite outgrowth (collagen-1 814±172 μm, Matrigel 3.723±327 μm, hpS 3.982±442 μm, n=6) on both coatings were significantly increased compared to the collagen-1 control, but no significant difference between Matrigel and hpS could be detected (FIG. 11C).

### 3D Biocompatibility of hpS

### Fibrinogen hpS Tris-NaCl Mix

HUVEC cells seeded in a fibrinogen/hpS mix formed a randomly orientated cell network, whereas HUVEC seeded in fibrin clots solidified with thrombin, no HUVEC network formation was observed (FIG. 12A). The microstructure of fibrinogen/hpS on SEM analysis showed a higher porosity in the hpS Tris-NaCl/fibrinogen clot when compared to the traditional fibrinogen/thrombin clot (FIG. 12B). In order to assess the feasibility of 3D organoid studies in a hpS/fibrin clot, primary colon organoids were cultivated in Matrigel or a hpS/fibrin gel. Organoids of various diameter sizes from 90-240 μm were observed in both gels. Microscopical images after 5 days of culture, scale bar=200 μm, FIG. 12C).

### Discussion

In the here presented study we introduced the isolation of an effective method to isolate hpS (consisting of multiple proteins) from full term human placenta, as a novel platform for a human-material-based technology for TERM.

Matrigel is originally extracted using a Tris 2 M urea buffer. Various authors also used 2 M urea to isolate bioactive ECM from xenogenic tissues. Uriel and colleagues used Tris 2 M urea to isolate pro-angiogenic ECM gels for in vitro studies from dermis or fat tissue, with an additional dispase treatment performed to lower the DNA content to a final yield of 183.7±10.2 ng/mL.[3] This step could be easily integrated in our presented isolation method to significantly lower the remaining DNA in hpS as well, however, may have also an influence on its final bioactivity. Moore and colleagues used urea buffers ranging from 4 to 15 M, to isolate a pro-angiogenic protein fraction from human placenta. However, urea is an endogenous product of protein and amino acid catabolism primary present in liver tissue, and, the cancerogenic potential of urea has also still not been adequately assessed, due to relatively few studies that have tested the toxicokinetics of exogenous urea in clinical studies to date.

Due to all these issues, Tris 0.5 M NaCl buffers were used in our experiments to isolate hpS, which are reported to preserve higher amounts of angiogenic cytokines compared to Tris-urea buffers if used for the preparation of tissue isolates.

On average, 300-400 mL of liquid hpS was extracted from one single placenta weighing around 500 g. Hence, our substrate could be used as a coating, injected into tissues or soaked into any preexisting porous 3D materials for various cell culture applications.

The total protein concentration of hpS using a Tris 2 M urea buffer was significantly higher when compared to the Tris 0.5 M NaCl buffer, which might be the result of the higher ionic density. For instance, Moore and colleagues used a Tris 4 M urea buffer to yield similar protein content to Matrigel (around 15-20 mg/mL). Hence, higher ionic densities yield higher amounts of extracellular matrix proteins. But in the same way, they also seem to lower the amounts of residual bioactive growth factors (see FIG. 3). No significant differences of GAGs were detected in both hpS extracts when compared to native tissues. Using SDS PAGE, a heterogenic variety of separate protein bands ranging up to around 500 kDa were assessed in hpS Tris-NaCl, which may represent an accurate mimicry of the fully diversity of non-cellular physiologic human tissue (ECM), whereas Matrigel from tumors is composed of less proteins.

Collagen-1 was only detectable in urea-enriched buffers (Matrigel, hpS Tris-urea), but not on hpS Tris-NaCl, as determined by Western blot analysis and total amino acid analysis.

On angiogenesis arrays, higher amounts of various angiogenesis related proteins was assessed using the isolation protocol based on a Tris 0.5 M NaCl buffer, when compared to the use of a Tris 2 M urea buffer, to extract hpS. Angiogenin, the most prevalent chemokine in hpS, was also the most prevalent chemokine using a Tris 4 M urea buffer in literature, but only relatively low levels of other angiogenic proteins were found. Other authors using 0.5% SDS to extract ECM from human placenta and showed relatively high amounts of bFGF, TIMP-2, hepatocyte growth factor (HGF) or IGF binding proteins (IGFBP-1), but only relatively low levels of angiogenin were found.[4]

In this regard, beside angiogenin, a heterogeneous mixture of other angiogenic growth factors and chemokines led to the observed gfpHUVEC network formation on hpS. For instance, laminin-111 promotes angiogenesis in synergy with FGF-1 by gene regulation in endothelial cells. Leptin, an endocrine hormone, stimulates angiogenesis in synergistic effect with FGF. Another prominent example is VEGF, known to play fundamental roles in early phase of neovascularization (tip cell), whereas angiopoietin is associated to late stage neovascularization (maturation of blood vessels).

Interestingly, hpS Tris-NaCl also contains thrombin, which upon mixing with fibrinogen can be used to form stable fully-human 3D fibrin scaffolds (clots). In addition, hpS Tris-NaCl has also antimicrobial properties dependent on the bacterial strain. The antibacterial effect was most prominent in S. carnosus, whose growth was almost completely inhibited by hpS Tris-NaCl. Interestingly, other strains were not affected by hpS Tris-NaCl. However, the underlying mechanism has not been investigated so far.

The total amino acid analysis was used to identify the content of amino acids suitable for chemical crosslinking with other materials. The amino acid composition of hpS Tris-NaCl displayed relatively high contents of amino acids with modifiable side groups (about 20 mol % NH₂/COOH residues) for functionalization strategies such as anhydride (e.g., norbornene anhydride), NHS activation (e.g., allylglycidyl), or vinyl esters.

Beside the characterization of the isolates we performed various experiments to show their usability in 2D as well as 3D cell culture applications. In our 2D in vitro assays, the cell network characteristics highly depended on the numbers of cells seeded, but not on different placenta (weighing each approximately 500 g). In all experiments performed, a significantly higher network complexity was observed using hpS coatings (p<0.001) when compared to Matrigel coatings. For instance, the mean tube length using hpS coatings reflects the physiological appearance (close mesh, e.g., like in a retina), whereas the mean tube length is significantly longer when using Matrigel from tumor-materials (wide-mesh). The interconnected cell networks on hpS remained for around five days in vitro, even when only using minimally essential RPMI medium, whereas the cell networks on Matrigel develop faster, but also degrade faster, as reported in literature. There were no significant differences of the cell network characteristics observed on both hpS substrates, although the total protein content in Tris-NaCl is around 25% lower than Tris-urea, and it contains a different protein composition.

The physiological relevance of Matrigel as a cell culture substrate is often called into question, as assays performed on Matrigel may result in false positive and false negative research results. For instance, in vitro, endothelial, but also many non-endothelial cells types such as NIH3T3-fibroblasts, melanoma, glioblastoma, breast cancer or aortic smooth muscle cell lines are already reported to form interconnected networks when seeded on Matrigel. Therefore, we performed an experiment using gfpNIH3T3 fibroblasts. While these cells did not form networks on hpS, they spontaneously formed networks on Matrigel within the first 24 h, which again confirms that Matrigel can also provoke false positive or negative research results.

Using a physiological Tris 0.15 M NaCl buffer to precipitate hpS would substitute the remaining dialysis steps, however the protein concentration and the final in vitro bioactivity was low when compared to a Tris 0.5 M NaCl precipitation buffer. We could also show that hpS can also be used as a cell culture medium supplement. More studies are currently studied to assess its full potential as a medium supplement for various cell types.

After the 2D experiments we translated our findings to 3D approaches since they are known to mimic the in vivo situation more accurate, when compared to 2D in vitro techniques. Indeed, many new technologies have been explored over the last years to pattern vascular cells in 3D hydrogels, and to guide vascular organization via chemical or mechanical signals. In addition, various publications have shown that channeled hydrogels improve the vascularization rate in 3D matrices. Hence, various fabrication techniques have already been utilized to create channel networks in hydrogels including (1) removable structures, (2) 3D laser-assisted printing of photo-hydrogels or (3) planar processing such as layer-by-layer UV radiation and polymerization of hydrogels.

For our experiments, we mixed hpS with various natural proteins to form 3D hydrogels, to provide a useful material for many in vitro applications such as 3D cell culture, bio printing or perfused constructs.

For instance, in our 3D vasculogenesis studies, freeze-dried human fibrinogen, a clinically established product for decades, was mixed with hpS Iris-NaCl to induce a randomly-oriented vasculogenic cell network in 3D after around 8 days of in vitro culture, where as in traditional fibrin clots mixed with thrombin, no vasculogenic effects were observed within this time frame.

### CONCLUSION

In summary, an effective method to isolate multiple proteins with angiogenesis-inductive properties from healthy human placenta tissue (hpS) with various potential applications for TERM was established. This material could be used as a novel platform for a human-material-based technology, for various 2D and 3D in vitro assays and techniques, as a medium supplementation, and most probably also for clinical applications.

### REFERENCES

[1] Gilbert T W. Strategies for tissue and organ decellularization. J Cell Biochem. 2012; 113(March):2217-2222. doi:10.1002/jcb.24130 Arnaoutova I, Kleinman H K. In vitro angiogenesis: endothelial cell tube formation on gelled basement membrane extract. Nat Protoc. 2010; 5(4):628-635. doi:10.1038/nprot.2010.6
[2] Moore M C, Pandolfi V, McFetridge P S. Novel human-derived extracellular matrix induces in vitro and in vivo vascularization and inhibits fibrosis. Biomaterials. 2015; 49:37-46. doi:10.1016/j.biomaterials.2015.01.022
[3] Uriel S, Labay E, Francis-Sedlak M, et al. Extraction and assembly of tissue-derived gels for cell culture and tissue engineering. Tissue Eng Part C Methods. 2009; 15(3):309-321. doi:10.1089/ten.tec.2008.0309
[4] Choi J S, Kim J D, Yoon H S, Cho Y W. Full-Thickness Skin Wound Healing Using Human Placenta-Derived Extracellular Matrix Containing Bioactive Molecules. Tissue Eng Part A. 2012; 19:120924061154007. doi:10.1089/ten.tea.2011.0738
[5] Gilbert T W. Strategies for tissue and organ decellularization. J Cell Biochem. 2012; 113(March):2217-2222. doi:10.1002/jcb.24130

1. A biologically active placenta-derived liquid substrate (hpS) containing extracellular matrix (ECM) proteins, cytokines and growth factors.

2. The liquid substrate of claim 1, wherein the content of cytokines and growth factors is increased when compared to Matrigel.

3. The liquid substrate of claim 1, wherein the growth factors are selected from the group consisting of angiogenin (ANG), angiostatin (PLG), basic fibroblast growth factor (bFGF), tissue inhibitor of metalloproteinases (TIMP), growth regulated protein (GRO), matrix metalloproteinase (MMP), angiopoietin (ANGPT), platelet endothelial cell adhesion molecule (PECAM), Leptin, interleukins (IL), RANTES (CCL5), tyrosine kinase-2 (TIE-2), urokinase plasminogen activator (uPAR), tumor necrosis factor-alpha (TNF-α), epidermal growth factor (EGF), granulocyte colony stimulating factor (G-CSF), monocyte chemotactic protein (MCP), interferon inducible T-cell α chemokine (I-TAC), monocyte chemotactic protein (MCP), epithelial neutrophil activating peptide 78 (ENA-78), I-309 (CCL1), endostatin, platelet-derived growth factor (PDGF), vascular endothelial growth factor (VEGF), interferon gamma (IFN-γ), insulin-like growth factor 1 (IGF-1), placental growth factor (PLGF), granulocyte macrophage colony stimulating factor (GM-CSF), transforming growth factor (TGF), and thrombopoietin (THPO).

4. The liquid substrate according to claim 1, wherein the extracellular matrix (ECM) proteins are selected from the group consisting of basal membrane proteins and a proteins from a blood lineage.

5. The liquid substrate of claim 4, wherein the basal membrane proteins are laminin-111 and collagen-4.

6. The liquid substrate of claim 4, wherein the protein from a blood lineage is thrombin.

7. The liquid substrate according to claim 5, wherein laminin-111 comprises about 90% of the liquid substrate's total protein content.

8. The liquid substrate according to claim 5, wherein collagen-4 comprises about 10% of the liquid substrate's total protein content.

9. The liquid substrate according to claim 1, wherein collagen-1 comprises less than 1% of the liquid substrate's total protein content.

10. The liquid substrate according to claim 1, further comprising one or more antimicrobial agents.

11. The liquid substrate according to claim 1, wherein the liquid substrate has a protein content in the range of 1.0 to 2.0 mg/mL, or 1.5 to 1.9 mg/mL, or 1.7 to 1.8 mg/mL.

12. The liquid substrate according to claim 1, wherein the liquid substrate further comprises natural polymers or synthetic polymers for solidification of the liquid substrate.

13. The liquid substrate according to claim 1, wherein said substrate does not gel at temperatures up to 37° C.

14. The liquid substrate according to claim 1, wherein the liquid substrate is obtained by a treatment with a non-denaturizing Tris NaCl buffer.

15. The liquid substrate of claim 14, wherein the treatment is carried out with Tris 0.5 M NaCl buffer.

16. A process for preparing a biologically active placenta-derived liquid human substrate (hpS) comprising the steps of:
a. providing a sample from human placenta tissue;
b. removing blood from said sample to obtain a crude extract;
c. solubilizing proteins in said crude extract using Tris-NaCl buffer;
d. separating solid materials from the solubilized protein extract mixture;
e. dialyzing the solubilized protein extract; and
f. obtaining the liquid substrate.

17. The process according to claim 16, wherein the extraction step is carried out using at least 0.2, 0.3, 0.4, 0.5, 0.6, 0.7, 0.8, 0.9, 1, 2, 3, 4, 5, or 6 M Tris-NaCl buffer.

18. The process according to claim 16, wherein the extraction step is carried out in the absence of a denaturizing agent.

19. The liquid substrate of claim 1, further comprising a natural and/or synthetic polymer to achieve solidification of the liquid substrate.

20-21. (canceled)

22. A method of cultivating cells, comprising the steps of:
adding the biologically active placenta-derived liquid substrate of claim 1 to a cell culture medium to produce a supplemented cell culture medium, and
cultivating cells with the supplemented cell culture medium.

23-24. (canceled)
